(19) United States
(12) Patent Application Publication
(10) Pub. No.: US 2003/0045699 A1
(43) Pub. Date: Mar. 6, 2003

(54) Membrane receptors for steroids and sterols

(76) Inventors: Bertram Weiss, Berlin (DE); Monika Lessl, Berlin (DE); Luisella Toschi, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/132,382

(22) Filed: Apr. 26, 2002

Related U.S. Application Data
(60) Provisional application No. 60/291,312, filed on May 17, 2001.

(30) Foreign Application Priority Data
Apr. 26, 2001 (DE) 10120337.3
Nov. 13, 2001 (DE) 10156597.6

(51) Int. Cl. C07H 21/02; C07H 21/04

(52) U.S. Cl. 536/23.1

(57) ABSTRACT

Four splice variants of a membrane receptor for steroids and sterols and their use are described.

The invention relates to nucleic acids that code for a membrane receptor for steroids and sterols, polypeptides and their use.

According to the standard model of steroid hormone action, the steroids bind to intracellular receptors that as ligand-activated transcription factors regulate the gene expression. It is common to refer to the direct action of the steroid hormones via their nuclear-position receptors to the transcription of target genes as genomic effects. Genomic effects are characterized by their time-delayed entry, i.e., more than 5 minutes up to several hours after hormone administration (McEwen et al. 1978 in: Reichlin et al. Eds., The Hypothalamus, Raven Press, p. 255 ff).

In contrast to the genomic actions, “quick” non-genomic effects that occur within a few seconds or minutes after the administration of steroid hormones and whose cause cannot lie in regulation of transcription have been observed for many years. Biochemical indicators for these effects are changes of membrane potential and/or the influencing of ion channels in the cell membrane (Watson et al. 1999, Proc Soc Exp Biol Med 220, 9 ff). These effects could be mediated by membrane-fixed receptors. Steroid effects that indicate the existence of a membrane receptor can also often be triggered by steroid derivatives that can no longer go through the plasma membrane by coupling to a bulky carrier molecule (for example serum albumin) and, made visible by fluorescence microscopy, bind to the cell surface. These results, just like binding studies with cell membranes and immune staining, indicate the existence of a membrane-fixed steroid receptor.

An example of a non-genomic action is the modulation of the γ-aminobutyric acid (GABA)-receptor by progesterone. This signal path is responsible for the mediation of the anesthetic action of progesterone (Covey, D. F. et al. 2000 J Pharmacol Exp Ther 293, 1009). Another example of a non-genomic progesterone action is the readmission of meiosis in amphibian oocytes. The progesterone-induced readmission of meiosis comprises the activation of cAMP-dependent protein kinases and the activation of MAPK signal paths (Palmer, A. et al. 2000 Prog Cell Cycle Res 4, 131 ff) and results in “germinal vesicle breakdown,” GVBD (Masui, Y. et al. 1971, J Exp Zool 177, 129 ff). While some findings suggest that the effects could be mediated via a membrane receptor for progesterone (mPR), the newest studies suggest that the nuclear-position PR could interact with tyrosine kinase signal paths (Tian, J. et al. 2000, Proc Natl Acad Sci USA 97, 14358 ff; Bayaa, M. et al. 2000, Proc Natl Acad Sci USA 97, 12607).

In a large number of publications, non-genomic estrogen effects are described for a great number of tissue and culture cells. Within a few minutes, the administration of estrogen triggers a number of effects: intracellular Ca²⁺ “spikes” (Mendoza, C.; Soler, A.; Tesarik, J. 1995, Biochem Biophys Res Commun 210: 518-523), stimulation of adenylate cylase (Aronica, S. M.; Kraus, W. L.; Katzenellenbogen, B. S., 1994, Proc Natl Acad Sci 91: 8517-21), and activation of NO-synthetase and phospholipase C (Le Mellay, V.; Grosse, B.; Lieberherr, M. 1997, J Biol Chem 272: 11902-11907).

Despite the extensive literature on non-genomic effects of steroid hormones, a membrane-fixed receptor for steroids in mammals previously could not be clearly identified. Should such a receptor exist, it would open up a quite new path for the development of medications. The provision of such a receptor is therefore the object of this invention.

The problem was solved by provision of a nucleic acid that comprises

a. The nucleotide sequence that is shown in Seq ID NO 1, NO 3, NO 5 and NO 7,

b. A nucleotide sequence that corresponds to the sequences of a. within the scope of the degeneration of the genetic code, or

c. A nucleotide sequence that hybridizes with the sequences of a. or b. under stringent conditions.

The term “hybridization under stringent conditions” according to this invention is defined in Sambrook et al. (Molecular Cloning, A Laboratory Manual, Cold Spring Harbor Laboratory Press, 1989). A stringent hybridization exists, for example, when a hybridization signal is observed after washing for 1 hour with 1× SSC and 0.1% SDS at 50° C., preferably at 55° C., especially preferably at 62° C. and most preferably at 68° C., especially for 1 hour in 0.2× SSC and 0.1% SDS at 55° C., preferably at 62° C. and most preferably at 68° C. The nucleic acids, which hybridize under these conditions with the nucleic acid that is shown in Seq. ID NO 1 and/or 3 or a nucleotide sequence that corresponds to this sequence in the scope of the degeneration of the genetic code, are also the subject matter of this invention.

Nucleic acids can produce single- or double-stranded DNA, e.g., cDNA, or RNA, e.g., mRNA, cRNA, or pre-mRNA.

The nucleic acids that are shown in SEQ ID NOS 1, 3, 5 and 7 code for a membrane receptor for steroids and sterols. The four nucleic acids are splice variants of the same gene. The variants that are shown in SEQ ID NO 3 and SEQ ID NO 7 in each case have an insertion in the 5′ area of the sequence.

The sequences that are shown in Seq ID NOS 5 and 7 respectively contain a deletion between two ankyrin domains.

The nucleic acids that comprise a protein-coding section of the nucleic acid sequence that is shown in Seq ID NO 1, 3, 5 or 7 are preferred. A protein-coding section of the sequence that is shown in Seq ID NO 1 is in the nucleotide range of 227 to 6271, a coding range of the sequence that is shown in SEQ ID NO 3 is in the nucleotide range of 360 to 6482, the sequence that is shown in Seq ID NO 5 is in the nucleotide range of 227-6993, and the sequence that is shown in Seq ID NO 7 is in the nucleotide range of 360-6408.

The subject matter of the invention are also nucleic acids that code for a polypeptide with the amino acid sequence that is shown in Seq ID NO 2, 4, 6 or 8.

The nucleic acids according to the invention can be obtained from mammals, e.g., human cells, or from a cDNA library or a genomic library, which is obtained from, e.g., human cells. They can be isolated according to known techniques with use of short sections of the nucleic acid sequence that is shown in Seq ID NO 1, 3, 5 or 7 as hybridization probes or amplification primers.

In addition, the invention relates to polypeptides that are coded by a nucleic acid according to the invention. These polypeptides have the function of a steroid receptor.

In addition, the subject matter of the invention are polypeptides that comprise the amino acid sequence that is shown in Seq ID NO 2, 4, 6 or 8.

The polypeptides according to the invention can be recombinant polypeptides; natural, isolated polypeptides; or synthetic polypeptides.

The polypeptides according to the invention contain various domains: 12 so-called “leucine-rich repeats” (LRR), 3-ankyrin domains (ANK) and a kinase domain. These polypeptides are membrane-associated. After the binding of a steroid or sterol, the kinase domain phosphorylates serine and threonine radicals of proteins and thus initiates an intracellular signal cascade. The polypeptides according to the invention also contain a so-called “island” domain, which comprises 600 amino acids and is found between the 11th and 12th leucine-rich repeat domain of the polypeptide according to the invention. This domain is involved in the steroid or sterol bond.

The invention also relates to polypeptides that comprise the kinase domains of the polypeptides according to the invention or portions thereof. The kinase domain is found in, for example, the amino acid range of 1270 to 1565, whereby the numbering relates to the sequence that is shown in Seq ID No 2.

The subject matter of the invention are also polypeptides that comprise the island domain of the polypeptides according to the invention. The latter contains a steroid or sterol binding site.

The polypeptides of Seq ID NOS 2, 4, 6 and 8 according to the invention are preferably expressed in tissues, of which it is known that they are steroid hormone-sensitive. Examples are ovaries, fallopian tubes, prostates and blood.

The polypeptides according to the invention or partial areas thereof (peptides) can be used for the production of antibodies. For the production of polyclonal antibodies, the polypeptides or peptides can be bonded to, e.g., KLH (Keyhole Limpet Hemocyanin), and animals, e.g., rabbits, can be sprayed. They can also be used for the production of monoclonal antibodies. For antibody production, a polypeptide or peptide according to the invention or a mixture of several peptides according to the invention can be used. In this case, the production of antibodies is carried out according to standard processes as they are described in, e.g., Kohler, G. and Milstein, C., Nature 1975, 256, 495-497 and Nelson, P. N. et al., Mol. Pathol. 2000, 53, 111-117.

The subject matter of the invention are also the antibodies that are directed against a polypeptide according to the invention.

The antibodies according to the invention can be used for detecting the polypeptides according to the invention. This can be carried out by, e.g., immunohistochemistry. The antibodies according to the invention can also be used in other immune tests, such as, e.g., an ELISA (enzyme linked immunosorbent assay) or in radioimmuno tests. Thus, the concentration of polypeptide according to the invention can be detected in tissue or cell extracts.

The detection of the expression of the polypeptide according to the invention can also be carried out via the detection of mRNA in the cells. The subject matter of the invention is therefore also the use of a probe with nucleic acid sequences that are complementary to the nucleic acid sequences, which code for the peptides according to the invention, for the production of a reagent for detecting the presence of the mRNA according to the invention in cells. A probe is a short strand of DNA with at least 14 nucleotides. The probes according to the invention can be used in, e.g., a Northern Blot Analysis. This method is described in, e.g., Sambrook, J. et al., 1989, Cold Spring Harbor Laboratory Press. Other methods for detecting RNA are in situ hybridization, RNAse Protection Assay or PCR.

In addition, the subject matter of the invention are vectors that contain at least one copy of a nucleic acid according to the invention. Vectors can be prokaryotic or eukaryotic vectors. Examples of vectors are pPRO (Clontech), pBAD (Invitrogen), pSGS (Stratagene), pCl (Promega), pIRES (Clontech), pBAC (Clontech), PMET (Invitrogen), and pBlueBac (Invitrogen). The nucleic acids according to the invention can be introduced into these vectors with the methods that are known to one skilled in the art. The nucleic acids according to the invention are preferably found in connection with expression signals, such as, e.g, promoters and enhancers, in the vector.

The invention also relates to cells that are transfixed with a nucleic acid sequence according to the invention or with a vector according to the invention. As cells, e.g., E. coli, yeast, Pichia, Sf9, COS, CV-1 or BHK can be used. These cells can be used for the production of the polypeptides according to the invention or for test systems.

A subject of the invention is the use of the polypeptides according to the invention or the nucleic acids that code for them as a target substance for the production of an agent for treating sterol-dependent diseases or steroid hormone-dependent diseases.

The invention includes in particular the use of

a. A nucleic acid according to the invention,

b. A polypeptide according to the invention, or

c. A cell according to the invention for identifying effectors of a polypeptide according to the invention. Effectors are substances that have an inhibitory or activating effect on the polypeptide according to the invention and are able to influence the steroid receptor function of the polypeptides according to the invention.

In addition, the invention relates to a test system for identifying effectors of a polypeptide according to the invention, whereby a polypeptide according to the invention is incubated as a complete or partial sequence thereof with a steroid, a steroid analog or a sterol, and the amount of bound steroid, steroid analog or sterol is detected. As a partial sequence, e.g., the area that comprises the “leucine-rich repeats” and the island domain or shorter strands thereof can be used. The binding of the effectors can be measured, e.g., by labeled substances being used and the labeling (e.g., radioactivity or fluorescence) being measured. A displacement reaction can also be measured, however, by a binding labeled steroid being used and the displacement of the labeling by the receptor being measured after the addition of the substances to be tested.

This binding test can also be performed with a cell according to the invention that contains the polypeptide according to the invention. In addition to the binding of the substances that are to be tested, an intracellular effect can also be measured, such as, e.g., a change in Ca²⁺ concentration. This variant of the test system thus also provides an indication of a functional activity of the effector.

The subject matter of the invention is also a test system for identifying inhibitors of the kinase function of the polypeptide according to the invention, whereby a polypeptide according to the invention or its kinase domain is brought into contact with the substances to be tested, and the kinase activity is determined. In this case, the kinase activity is determined with and without the substance to be tested, and the extent of inhibition is determined.

The effectors of the sterol or steroid receptor function of the polypeptide according to the invention can be used for treating sterol-dependent diseases or steroid hormone-dependent diseases. Examples in this respect are perimenopausal and postmenopausal symptoms, diseases of the urogenital tract, ovarian dysfunction, osteoporosis, benign prostate hyperplasia, cardiovascular diseases, vascular diseases, neurodegenerative diseases, inflammatory diseases, and diseases of the immune system. In addition, these effectors can be used for treatment of female and male infertility.

It has been found that the polypeptide according to the invention binds the endogenous substance FF-MAS (meiosis-activating substance that consists of follicular liquid). FF-MAS induces the meiosis of female germ cells and is therefore an essential factor of female fertility. With the aid of a test system according to the invention, substances can be identified that inhibit the effect of FF-MAS on the receptor. These substances inhibit the fertility and can therefore be used as agents for birth control. Substances that activate the receptor and therefore act analogously to FF-MAS can also be identified with the test system according to the invention. These substances act to promote fertility and can either be administered to women who suffer from fertility disorders or they can be added in vitro to an oocyte culture. The thus prepared oocytes are then used for in vitro fertilization.

In addition, the invention relates to a process for the preparation of a pharmaceutical agent, whereby

a. Substances are brought into contact with a test system according to the invention,

b. The action of the substances on the test system is measured in comparison to controls,

c. A substance that shows a modulation of the activity of the polypeptides according to the invention in step b. is identified,

d. And the substance that is identified in step c. is mixed with the formulation substances that are commonly used in pharmaceutics.

Activity of the polypeptide according to the invention is defined as the steroid-binding property and/or the kinase activity of the polypeptide.

A substance that is identified by a process according to the invention can optionally be optimized relative to metabolic stability, activity in a test system according to the invention and/or bio-availability. Methods that are standard in chemistry can be used in this respect.

### DESCRIPTION OF THE FIGURES

FIG. 1 shows the domain structure of the polypeptides according to the invention. The structure begins to the left with the N-terminus.

FIG. 2 shows the result of an electronic Northern Blot Analysis.

FIG. 3 shows a sequence comparison of the DNA of all 4 splice variants. Here, the designation

SINGER_KINASE_SP1B corresponds to Seq ID No 5,

SINGERKINASE_SP2B corresponds to Seq ID No 7,

spliceVariant_{—}2a corresponds to Seq ID No 3 and

singerKinase_sp1a corresponds to Seq ID No 1.

FIG. 4 shows a comparison of the amino acid sequences of all 4 splice variants. The designations are explained under FIG. 3.

FIG. 5 shows the distribution of the RNA according to the invention in various human tissues. For the amplification, the following primers were used: 5′ gtatgaactgtgctgtgggaag 3′ (4928) and 5′ gatgtaccactccaccacctacc 3′ (5532). A Northern Blot is shown.

In the left picture, 1 refers to the spleen, 2 refers to the thymus, 3 refers to the prostate, 4 refers to the testis, 5 refers to the ovary, 6 refers to the small intestine, 7 refers to the colon, and 8 refers to peripheral blood leukocytes. In the right picture, 1 refers to the brain, 2 refers to the heart, 3 refers to the skeletal muscles, 4 refers to the colon, 5 refers to the thymus, 6 refers to the spleen, 7 refers to the kidney, 8 refers to the liver, 9 refers to the small intestine, 10 refers to the placenta, 11 refers to the lung, and 12 refers to peripheral blood leukocytes. The arrow shows the position of the strip that corresponds to the RNA according to the invention.

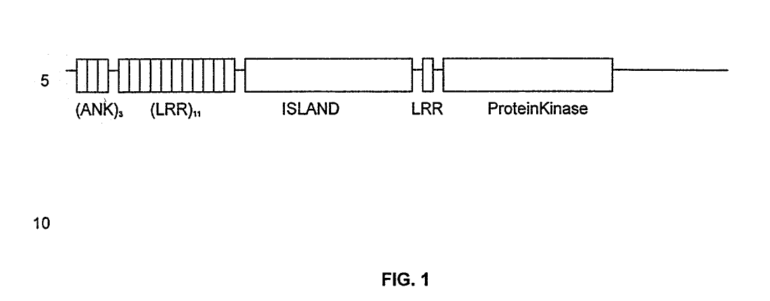

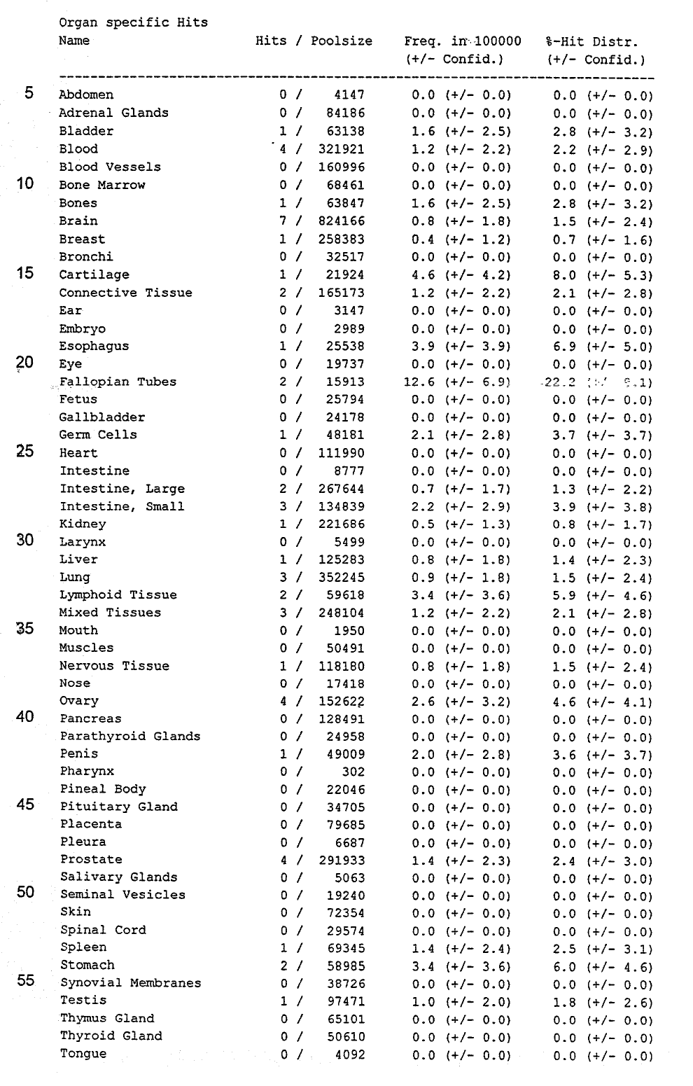

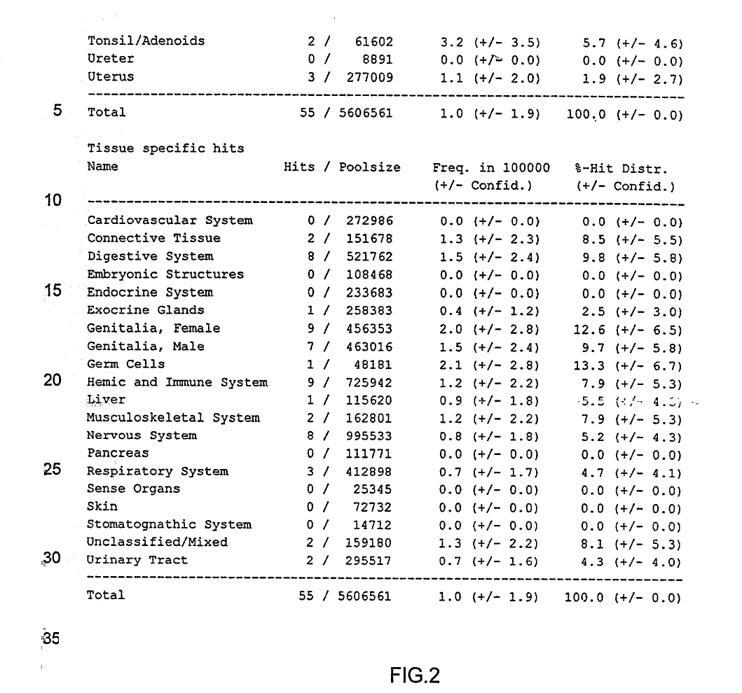

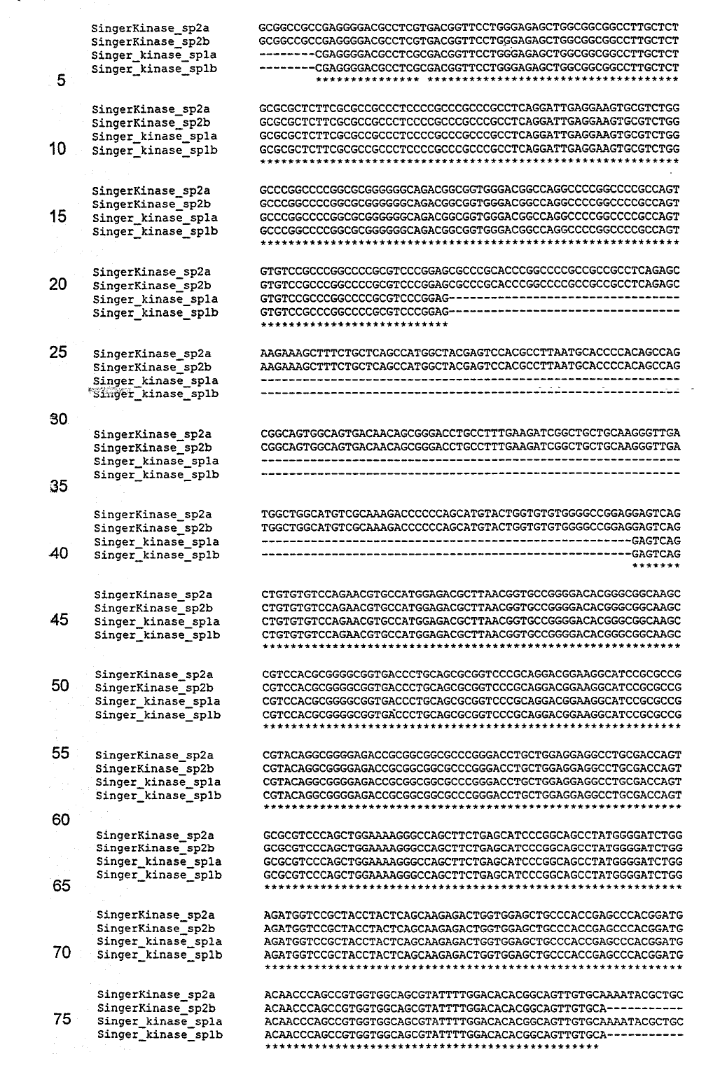

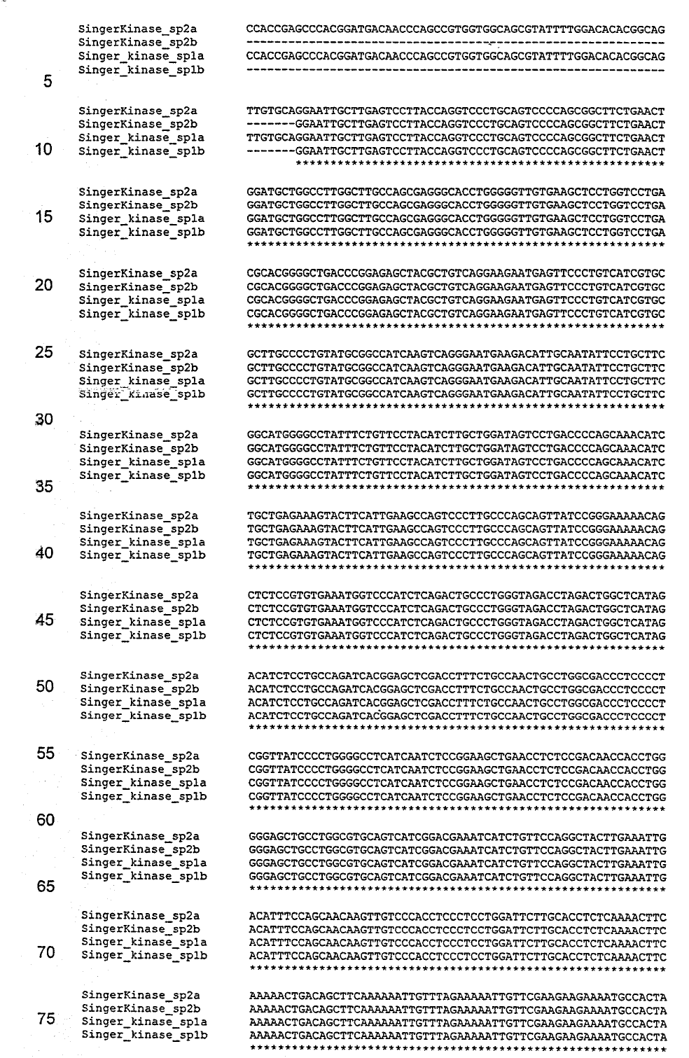

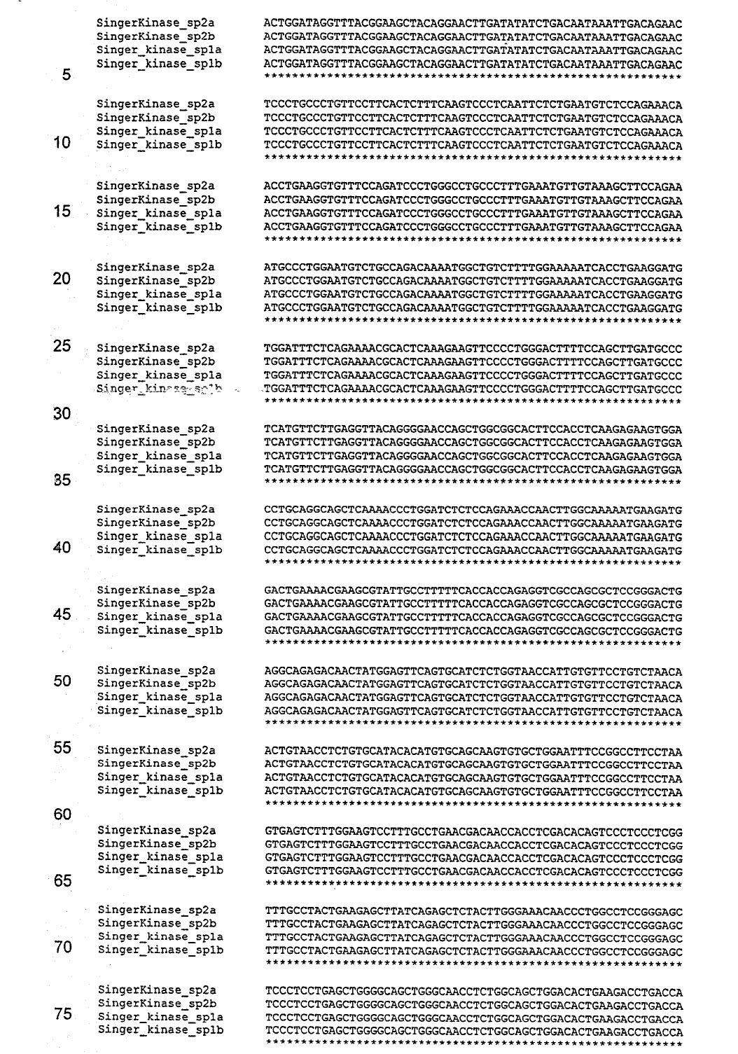

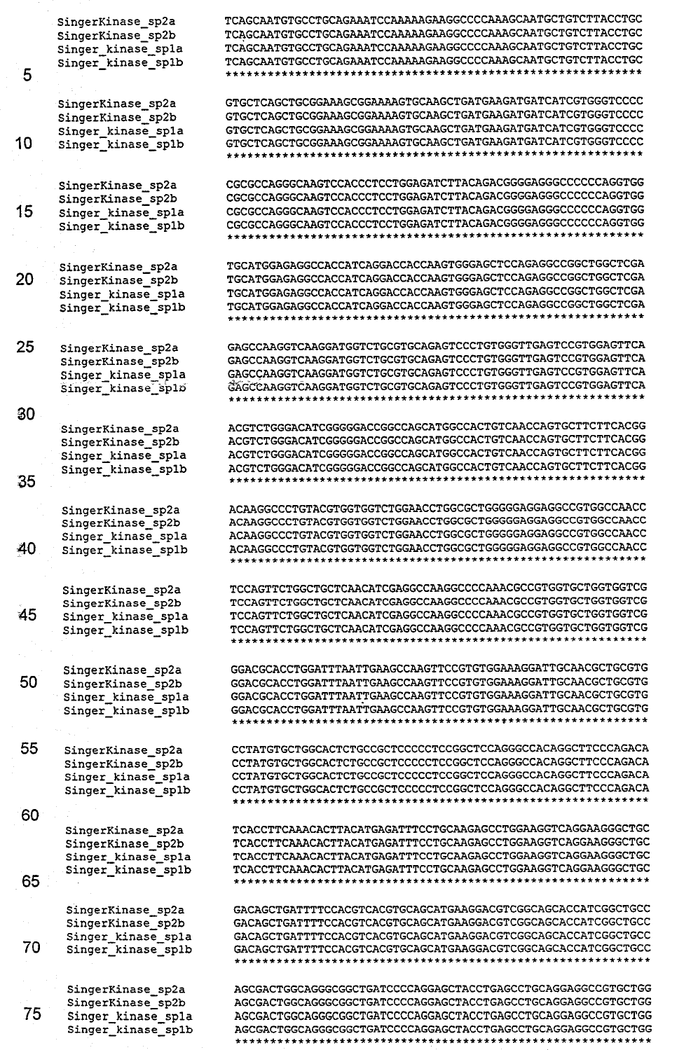

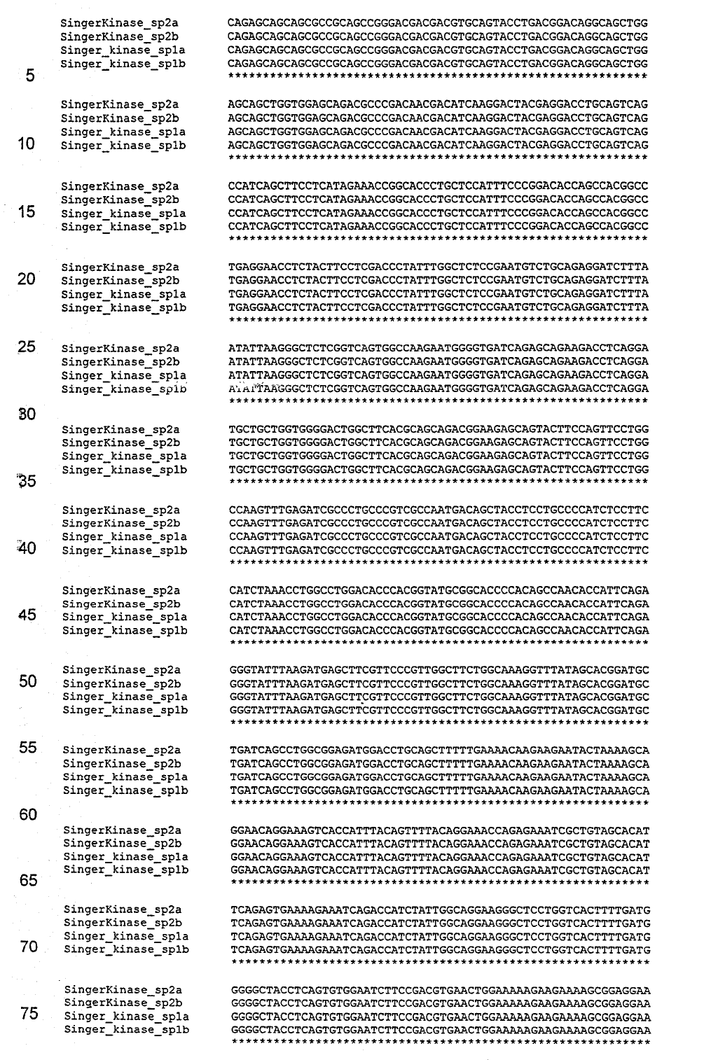

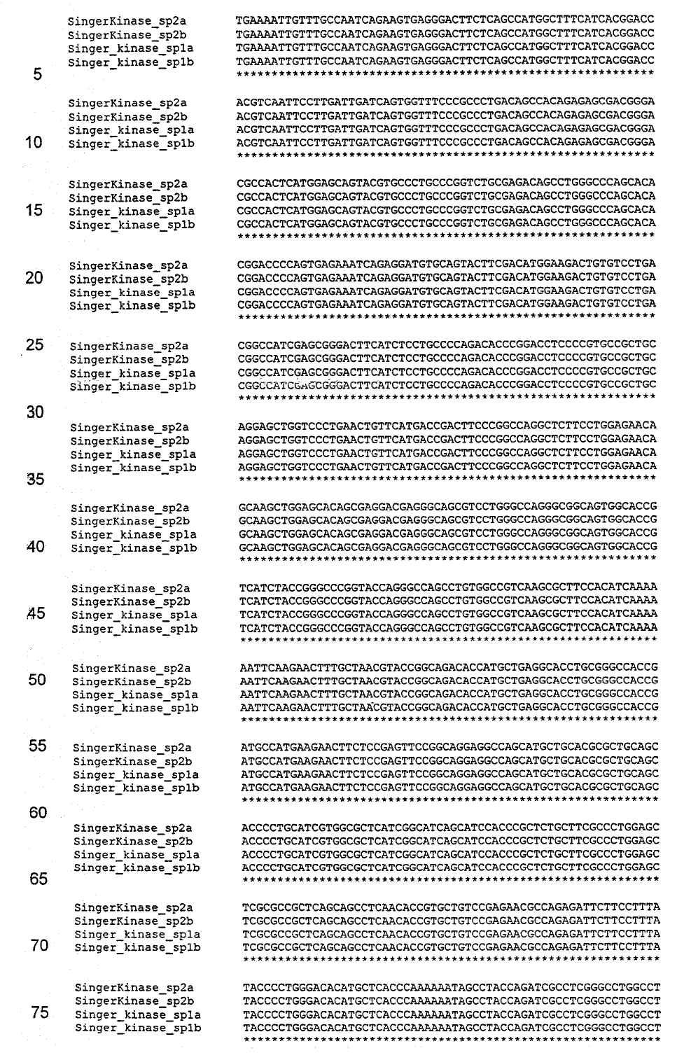

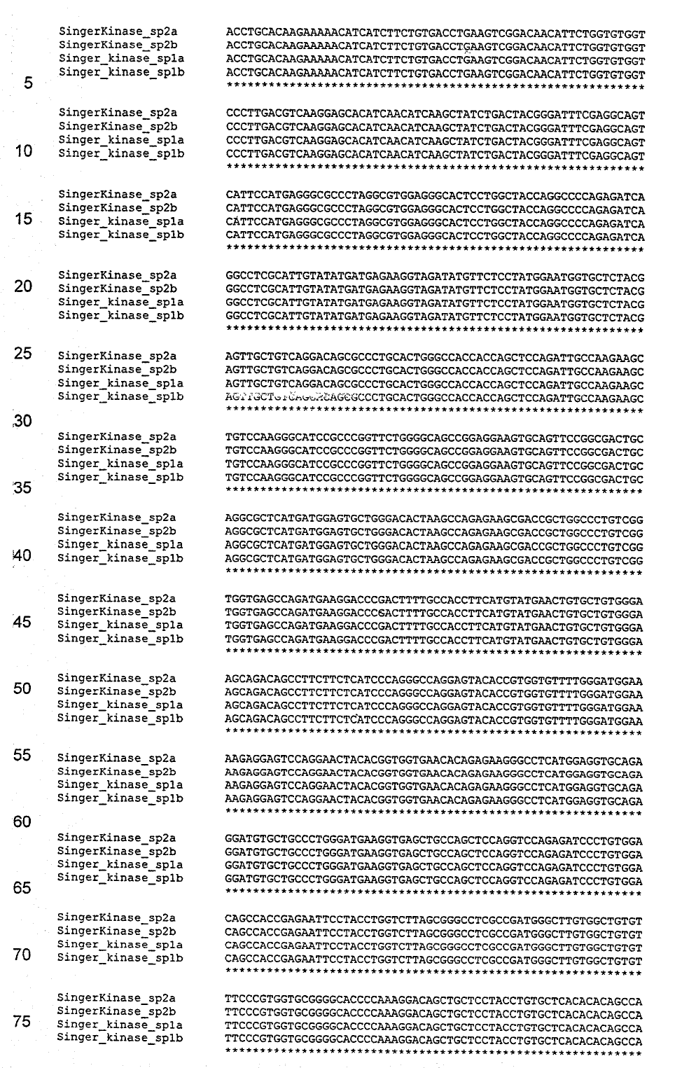

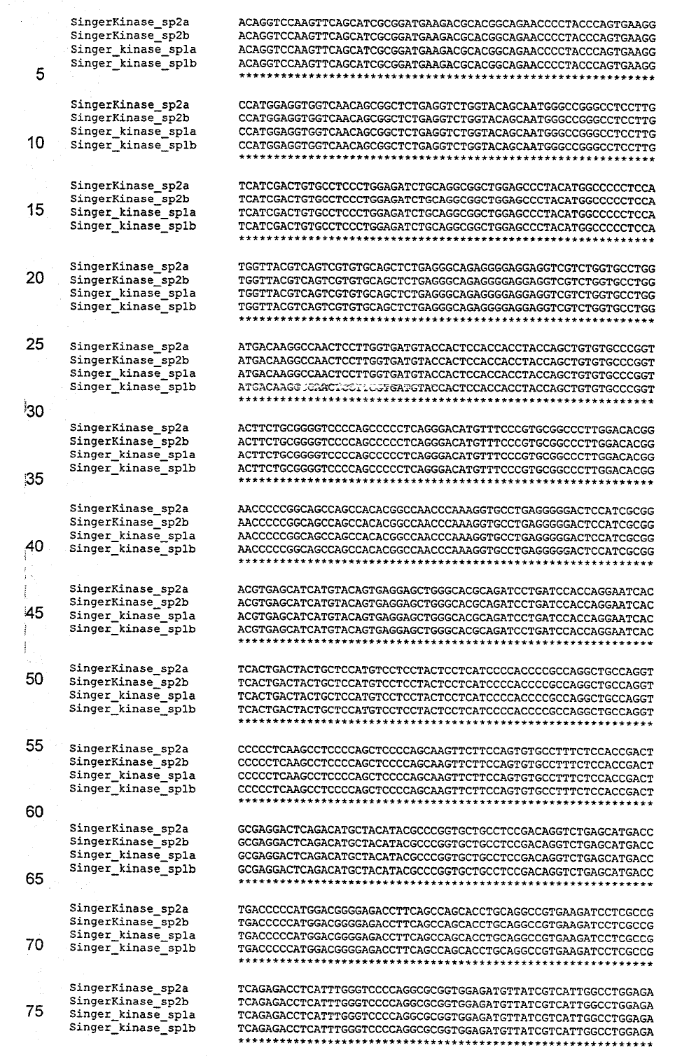

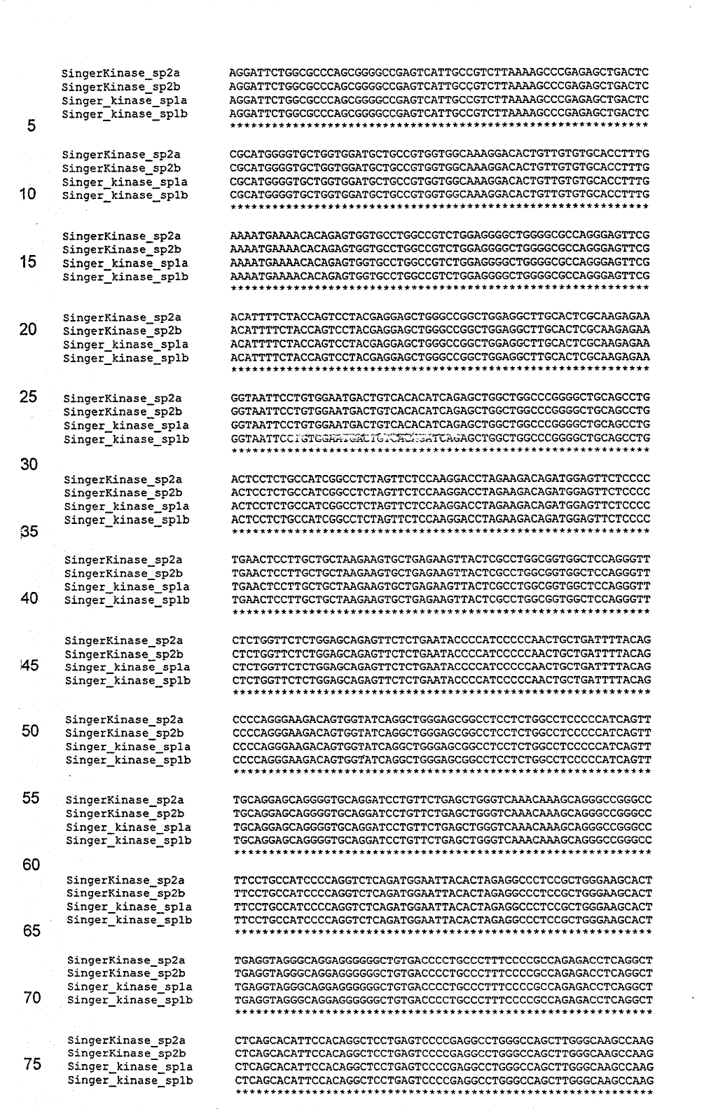

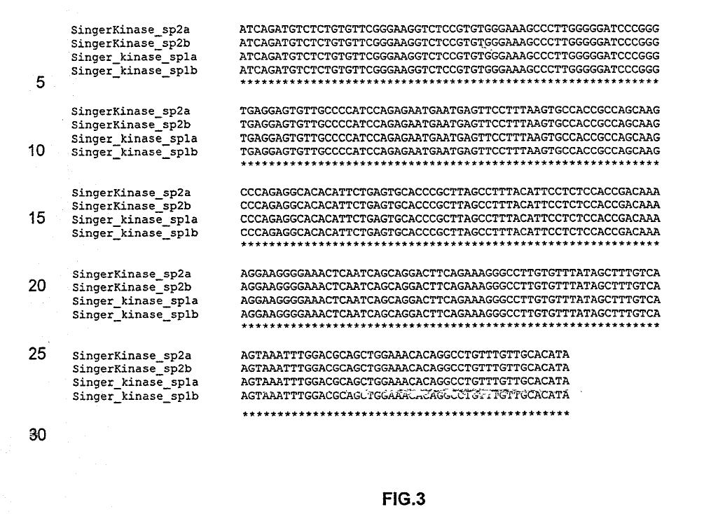

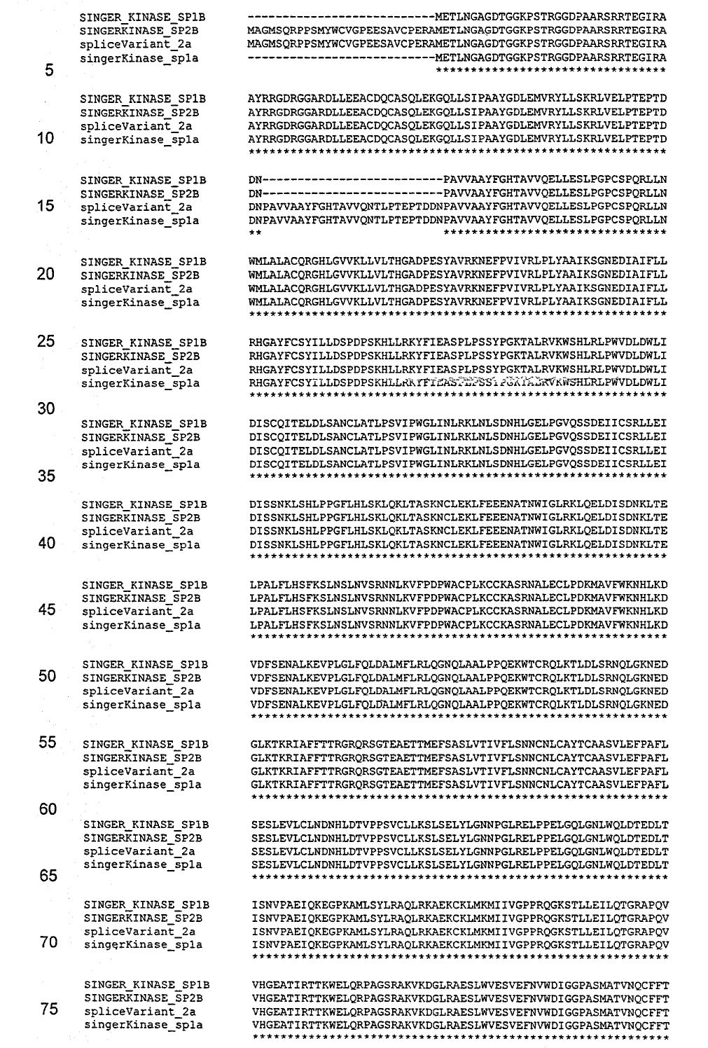

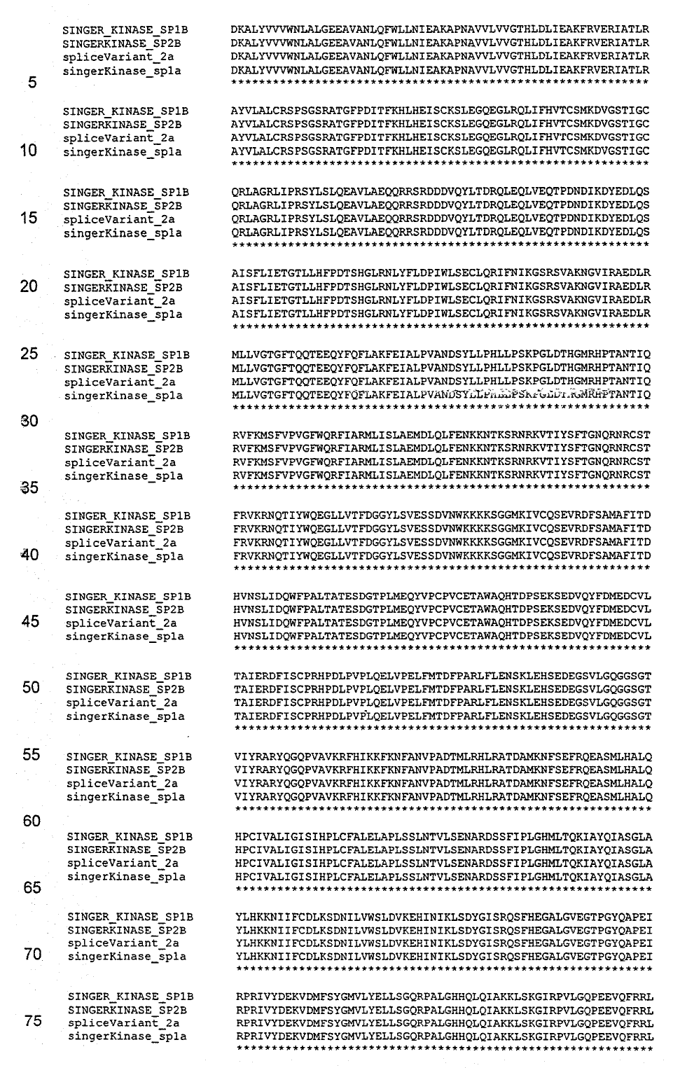

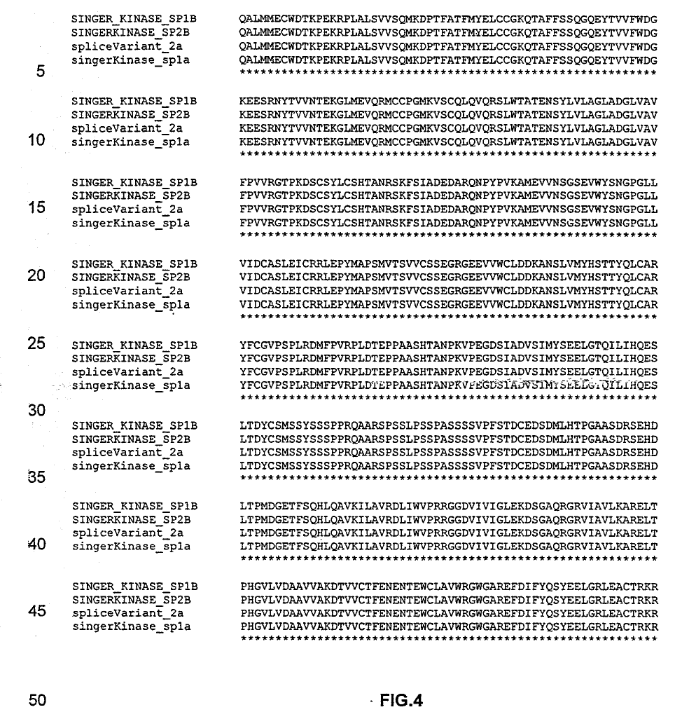

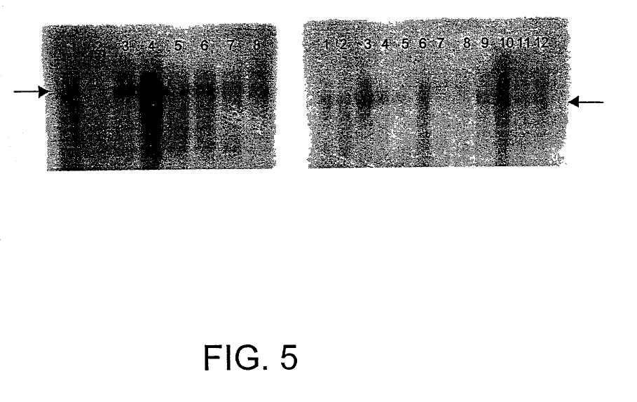

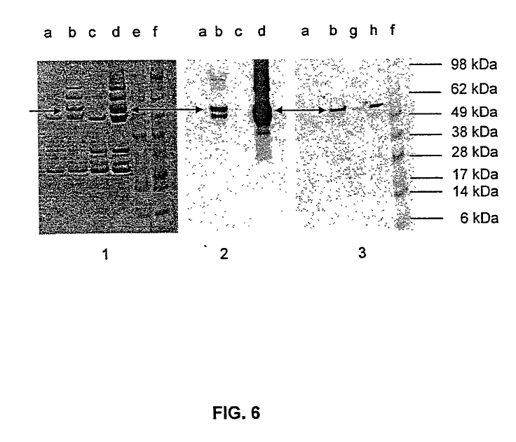

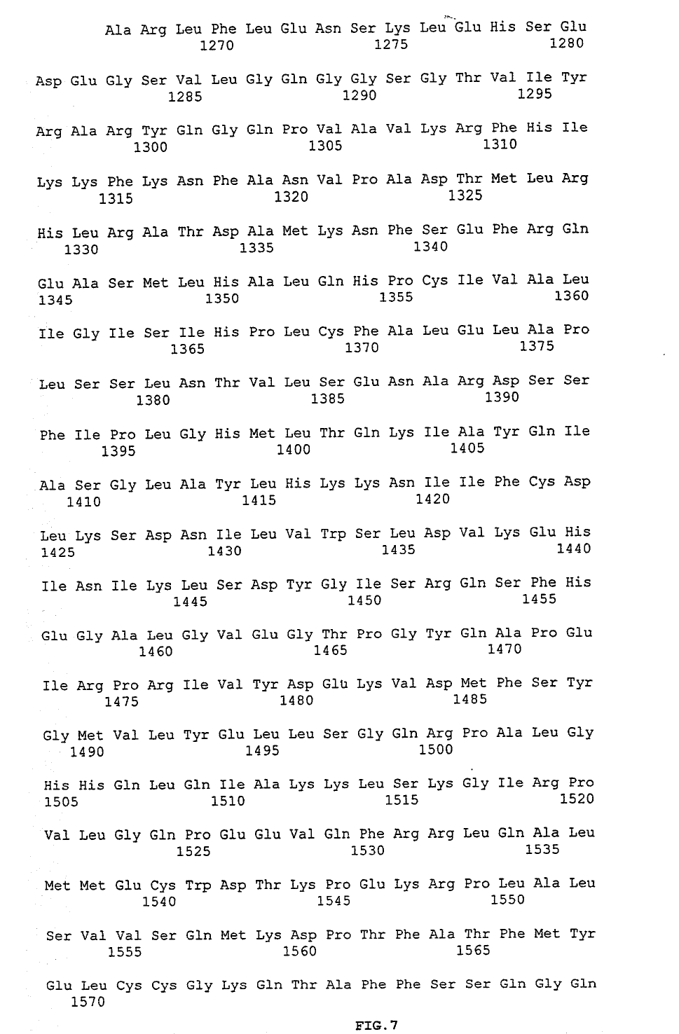

FIG. 6 shows the expression and auto-phosphorylation of the kinase domains. In FIG. 1, a polyacrylamide gel is shown after protein staining with Coomassie G-250; in FIG. 2, the corresponding autoradiogram (³³P) in shown, and in FIG. 3, a Western Blot is shown. Here: a refers to glutathione eluate (control), b refers to glutathione eluate (GST-SIN2), c refers to glutathione sepharose with bonded proteins (control), d refers to glutathione sepharose with bonded proteins (GST-SIN2), e+f mean molecular weight markers (SeaBlue), g refers to the SDS-eluate of the glutathione sepharose (control), and h refers to the SDS-eluate of the glutathione sepharose (GST-SIN2). The test conditions are described in Examples 3 and 4. The Western Blot was produced with anti-GST-antibodies (goat serum) as a first antibody and anti-goat IgG-alkaline phosphatase conjugate as a second antibody. The arrow marks the position of the GST-kinase fusion protein.

FIG. 7 shows the amino acid sequence of the fusion protein that consists of glutathione-S-transferase and the amino acids Ala1267-Leu1570.

### EXAMPLES

The molecular biological methods that are used in the examples, such as, e.g., polymerase chain reaction (PCR), production of cDNA, cloning of DNA, sequencing of DNA, were performed as described in known textbooks, such as in, for example, Molecular Cloning, A Laboratory Manual (Sambrook, J. et al., 1989, Cold Spring Harbor Laboratory Press).

### Example 1

### Cloning of the Membrane-Bound Steroid Receptor

For the cloning, a cDNA bank from the human mammary gland (Clontech) was used. The cDNA of the splice variants according to the invention was obtained by means of PCR. To this end, the following primers were used:

| | | |
|---|---|---|
| N-ter- | 5′-ATGGAGACGCTTAACGGTGC-3′ | |
| minal | | |
| area | | |
| #3F | 5′-GTTCCCTGTCATCGTGCGCTTGCCCC-3′ | (790-815) |
| #4F | 5′-CCAGCAACAAGTTGTCCCACCTCCC-3′ | (1233-1257) |
| #5F | 5′-GGATCTCTCCAGAAACCAACTTGGC-3′ | (1729-1753) |
| #6F | 5′-CCTGGAGATCTTACAGACGGGGAGGG-3′ | (2269-2294) |
| #7F | 5′-CGTCGGCAGCACCATCGGCTGCCAG-3′ | (2824-2848) |
| #8F | 5′-CCTCCTGCCCCATCTCCTTCCATC-3′ | (3307-3330) |
| #9F | 5′-GAAATCAGAGGATGTGCAGTACTTC-3′ | (3880-3904) |
| #10F | 5′-CACCGTGCTGTCCGAGAACGCCAG-3′ | (4369-4392) |
| #11F | 5′-CCTTCATGTATGAACTGTGCTGTG-3′ | (4920-4943) |
| #12F | 5′-CCATGGTTACGTCAGTCGTGTGC-3′ | (5424-5446) |
| #13F | 5′-CCGACAGGTCTGAGCATGACCTG-3′ | (5886-5908) |
| #2R | 5′-TTACCTTCTCTTGCGAGTGCAAGCC-3′ | (6247-6271) |
| C-ter- | 5′`GAGAACTCTGCTCCAGAGAAC 3′. | |
| minal | | |

For the amplification of the splice variants, which are shown in Seq Id Nos 1 and 3, the primer 5′-CGGATGACAACCCAGCCGTGGTGG-3′ (579-602) was also used. The numbering indicated in parentheses relates to the sequence that is shown in Seq ID No 1.

### Example 2

### Expression of the Membrane-Bound Steroid Receptor

For the expression, the coding area was amplified by means of PCR and introduced into the baculovirus expression vector pBlueBac4.5/V5-His-TOPO (Invitrogen) or the eukaryotic expression vector pcDNA3.1/V5/His-TOPO (Invitrogen). To simplify detection and purification, a fusion with an His tag was carried out. After co-transfection of insect cells with the Bac-N-Blue DNA, recombinant viruses were produced that were identified by a PCR process. A phage stock was then applied and used in larger amounts for further transfections and production of the receptor. The purification of the His-tagged proteins was carried out on a nickel affinity column.

### Example 3

### Cloning and Expression of the Kinase Domain

The kinase domain was amplified within a sizeable sequence from a cDNA library (mammary gland and lung, Clontech) by PCR and cloned in the TOPO vector. From this vector, only the kinase domain is amplified out.

For expression of the recombinant kinase domain as a glutathione-S-transferase-fusion protein, the vector pGex-KT (contains thrombin interfaces between the proteins) is used, and for fusion with MBP, the vector pMal-TBVp3 (modified, also with thrombin interfaces) is used. Within the context of cloning, the plasmids are transformed for the time being into the E. coli strain XL-1 and then for the expression of proteins into the E. coli strain BL21. The transformation is always carried out by thermal shock. The cells are cultivated in LB medium with 200 μg/ml, whereby in the expression of MBP-fusion proteins, 0.1% glucose must always be present in the medium.

In the expression of proteins, the cells grow up to an OD₆₀₀ of 0.8-0.9, then the induction is carried out with 0.5 mmol of IPTG for 3 hours at 25° C. Cell decomposition takes place in the French Press. The decomposition buffer contains 20 mmol of Tris, pH 7.4, 150 mmol of NaCl, 1 mmol of DTT, 0.1 mmol of EDTA and protease inhibitor. To separate the insoluble components, an ultracentrifuging is carried out at 35000×g after the cell decomposition. The MBP-fusion proteins are then purified on amylose resin in the buffer (20 mmol of Tris, pH 7.4, 150 mmol of NaCl, 1 mmol of DTT, 0.1 mmol of EDTA and protease inhibitor). The GST-fusion proteins are purified on sepharose beads (buffer as above).

In another approach for the expression of the recombinant kinase domain as a glutathione-S-transferase-fusion protein, the eukaryotic expression vector pDEST27 (Invitrogen 11812-013) is used.

This vector carries the cDNA that codes the kinase domain according to the invention in the reader frame of the N-terminal upstream glutathione-S-transferase CDNA. This results in the expression of a fusion protein from glutathione-S-transferase and the amino acids Ala1267-Leu1570 (amino acid numbering according to Seq ID No 2).

For the expression of the recombinant kinase domain, HEK293EBNA cells (Invitrogen) are transiently transfixed with the kinase-domain-coding pDEST27 vector. To this end, HEK293EBNA cells are cultivated in the DMEM medium (plus 10% FCS, plus Glutamax) until subconfluence is completed. The cells in a flask are transfixed according to the standard protocol of the manufacturer by means of lipfectamine reagent (Invitrogen). 150 μl of reagent and 75 μg of pDEST27-kinase domain DNA are used for transfection per 150 cm² flask (=GST-SIN2). The cells of a second flask are not transfixed (=control).

The cells are harvested after 48 hours of incubation: the conditioned medium is discarded. The cells are lysed on ice (25 mmol of HEPES/NaOH, pH 7.3+1% NP-40+2 mmol of EGTA+2 mmol of EDTA+10 mmol of β-glycerophosphate+2 mmol of DTT+phosphatase inhibitor cocktail No. 1 (SIGMA)+phosphatase inhibitor cocktail No. 2 (SIGMA)+Completeₘᵢₙᵢ-EDTA (Roche)+200 mmol of NaCl), frozen, thawed and centrifuged. The cell supernatant is further used.

For binding the GST-fusion protein to glutathione sepharose, the cell supernatant is added to the glutathione sepharose, incubated and washed. 20 μl of the glutathione sepharose with the proteins bonded thereto is used directly in the kinase assay as described in Example 4 (=glutathione sepharose with bonded proteins). The residual glutathione sepharose is eluted with a buffer that contains reduced glutathione (=glutathione eluate).

### Example 4

### Detection of the Activity of the Kinase Domain (Auto-phosphorylation)

10 μl each of the purified protein solution (see Example 3, glutathione sepharose with bonded proteins and glutathione eluate) is mixed with 10 μl of kinase assay buffer (25 mmol of HEPES/NaOH, pH 7.3+10 mmol of MgCl₂+10 mmol of MnCl₂+2 mmol of DTT+phosphatase inhibitor cocktail Nos. 1 and 2 (SIGMA)+200 mmol of NaCl). The phosphorylation reaction is started by adding 1 μl each of 100 μM ATP+4 μCi γ[³³P]-ATP1. The reaction is carried out for 60 minutes at 30° C. The samples are separated in an SDS-PAGE, and the gel is stained with a Coomassie G-250 solution. After the gel is dried, an autoradiography is carried out.

### Example 5

### Test System for Finding Effectors (Binding Assay) Homogenization

Cells and tissue, in which the protein according to the invention is expressed, were homogenized in buffer 1 at 4° C. Buffer 1:0.02 mmol/l of tris-buffer, pH 7.5, 0.5 mmol/l of EDTA, 2 mmol/l of DTT, 20% glycerol, 0.4 M KCL, 20 mmol/l of molybdate, 0.3 μmol/l of aprotinin, 1 μmol/l of pepstatin, 10 μmol/l of leupeptin.

### Receptor Binding Studies

Receptor binding studies were performed with crude extracts in buffer 1, cytosol preparations (supernatant after centrifuging the crude extract at 105,000×g, 90 minutes) and microsome fractions of cells and tissues that express the protein according to the invention.

Crude extracts and cytosol fractions (0.04 ml) were incubated at 4° C. for 2 hours with 10 μl of radioactive steroid or sterol (concentration in the batch 10 nmol/l) either in the absence or in the presence of a 100× excess of a non-radioactive substance (for determining the unspecific bond). After the incubation of the crude extract or the cytosol fraction with the radioactive steroids or sterols, unbound steroids or sterols were adsorbed in activated carbon by being incubated with 0.25 ml of a suspension of dextran-coated charcoal in buffer 1 for 5 minutes at 4° C. After centrifuging (5 minutes, 15,000 g), an aliquot of the supernatant was removed, and the radioactivity was determined by means of a scintillation counter. The specific receptor bond was determined as the difference between the bound radioactivity in the absence and in the presence of a 100× excess of a non-radioactive compound. As radiolabeled steroids and sterols, ³[H]-estradiol, ³[H]-progesterone, ³[H]-corticosterone, ³[H]-cortisol, ³[H]-aldosterone, ³[H]-dihydrotestosterone, ³[H]-testosterone, ³[H]-pregnenolone, ³[H]-cholesterol, ³[H]-androsterone, ³[H]-dihydroandrosterone, ³[H]-calcitriol and ³[H]-FF-MAS were used.

### Example 6

### Kinase Assay

The peptide that contains the kinase domains is expressed in SF9 or COS cells and purified via affinity chromatography (tag). The purified peptide is incubated in 20 mmol of Mops, pH 7.5, 25 mmol of β-glycerophosphate; 5 mmol of EGTA; 0.04% NP40 and 0.2% BSA in the kinase assay for 30 minutes with ³³P-ATP in MgCl₂ and the substrate Bio-Lys-Lys-Leu-Asn-Arg-Thr-Leu-Ser-Val-Ala-OH. This substrate is phosphorylated in the presence of ATP on serine or threonine and can be detected with the aid of streptavidin beads. The reaction is completed with 50 μm of ATP, 50 μm of EDTA, 0.1% Triton X-100 (v/v) and simultaneously mixed with the streptavidin beads. The reaction batches must be left for at least 10 minutes and then centrifuged for 10 minutes. The determination of the phosphorylation is carried out by measuring the radioactivity. Staurosporine is used as a reference substance for inhibiting the kinase activity of peptides.

### Example 7

### Electronic Northern

Based on the number of ESTs for a certain tissue, an electronic Northern provides an indication to the expression strength of a certain gene in this tissue. First, the number of all ESTs for a tissue is identified in a data bank (named pool-size), and then the number of ESTs of this tissue, which correspond to the nucleic acid according to Seq ID No. 1, is determined (named hits). As a method, a so-called “blast search” is used (NCBI BLAST v. 2.0.10; Altschul et al., Nucleic Acid Res. 1997, 25, 3389-3402). A search is run in an EST-data bank (LifeSeqGold from Incyte).

1. Nucleic acid that comprises
a. The nucleotide sequence that is shown in Seq ID NO 1, Seq ID NO 3, Seq ID NO 5 or Seq ID No 7,
b. A nucleotide sequence that corresponds to the sequences of a. within the scope of the degeneration of the genetic code, or
c. A nucleotide sequence that hybridizes with the sequences of a. under stringent conditions.

2. Nucleic acid according to claim 1 that comprises a protein-coding section of the nucleic acid sequence that is shown in Seq ID NO 1, Seq ID NO 3, Seq ID NO 5 or Seq ID No 7.

3. Nucleic acid that codes for a polypeptide with the amino acid sequence that is shown in Seq ID NO 2, Seq ID NO 4, Seq ID NO 6 or Seq ID NO 8.

4. Polypeptide that is coded by a nucleic acid according to one of claims 1-3.

5. Polypeptide that comprises the amino acid sequence that is shown in Seq ID NO 2, Seq ID NO 4, Seq ID NO 6 or Seq ID NO 8 or portions thereof.

6. Polypeptide that comprises the kinase domain of the amino acid sequence that is shown in Seq ID NO 2, Seq ID NO 4, Seq ID NO 6 or Seq ID NO 8.

7. Use of a polypeptide according to claim 4, 5 or 6 or of portions of this polypeptide for the production of antibodies.

8. Antibody against a polypeptide according to one of claims 4 to 6.

9. Use of a probe with nucleic acid sequences that are complementary to the nucleic acid sequences according to claims 1-3 for the production of a reagent for detecting the presence of mRNA according to one of claims 1 to 3 in cells.

10. Vector that contains at least one copy of a nucleic acid according to one of claims 1-3.

11. Cell that is transfixed with a nucleic acid according to one of claims 1-3 or with a vector according to claim 10.

12. Use of a cell according to claim 11 for the expression of nucleic acid according to one of claims 1-3.

13. Use of
a. A nucleic acid according to one of claims 1 to 3,
b. A polypeptide according to one of claims 4 to 6 or
c. A cell according to claim 11 for identifying effectors of a polypeptide according to claim 4 or 5.

14. Use of a nucleic acid according to claims 1 to 3 or a polypeptide according to claims 4 to 6 as a target substance for the production of an agent for steroid hormone-dependent diseases.

15. Test system for identifying effectors of a polypeptide according to claim 4 or 5, whereby a polypeptide is incubated according to claim 4 or 5 with a steroid, a steroid analog or a sterol, and the amount of bound steroid, steroid analog or sterol is detected.

16. Test system according to claim 14 or 15 whereby the effectors are FF-MAS agonists or antagonists.

17. Test system for identifying inhibitors of the kinase function of the polypeptide according to claim 4 or 5, whereby a polypeptide is brought into contact according to claim 4 or 5 with the substances that are to be tested, and the kinase activity is determined.

18. Process for the preparation of a pharmaceutical agent, whereby
a. Substances are brought into contact with a test system according to claim 15, 16 or 17,
b. The action of the substances on the test system is measured in comparison to controls,
c. A substance that shows a modulation of the activity of the polypeptide according to claim 4 or 5 in step b. is identified,
d. And the substance that is identified in step c. is mixed with the formulation substances that are commonly used in pharmaceutics.


SEQUENCE LISTING (26 sequences)

SEQ ID NO: 1 - DNA, 7093 bp, Homo sapiens
cgaggggacgcctcgcgacggttcctgggagagctggcggcggccttgctctgcgcgctcttcgcgccgccctccccgcccgcccgcctcaggattgaggaagtgcgtctgggcccggccccggcgcggggggcagacggcggtgggacggccaggccccggccccgccagtgtgtccgcccggccccgcgtcccggaggagtcagctgtgtgtccagaacgtgccatggagacgcttaacggtgccggggacacgggcggcaagccgtccacgcggggcggtgaccctgcagcgcggtcccgcaggacggaaggcatccgcgccgcgtacaggcggggagaccgcggcggcgcccgggacctgctggaggaggcctgcgaccagtgcgcgtcccagctggaaaagggccagcttctgagcatcccggcagcctatggggatctggagatggtccgctacctactcagcaagagactggtggagctgcccaccgagcccacggatgacaacccagccgtggtggcagcgtattttggacacacggcagttgtgcaaaatacgctgcccaccgagcccacggatgacaacccagccgtggtggcagcgtattttggacacacggcagttgtgcaggaattgcttgagtccttaccaggtccctgcagtccccagcggcttctgaactggatgctggccttggcttgccagcgagggcacctgggggttgtgaagctcctggtcctgacgcacggggctgacccggagagctacgctgtcaggaagaatgagttccctgtcatcgtgcgcttgcccctgtatgcggccatcaagtcagggaatgaagacattgcaatattcctgcttcggcatggggcctatttctgttcctacatcttgctggatagtcctgaccccagcaaacatctgctgagaaagtacttcattgaagccagtcccttgcccagcagttatccgggaaaaacagctctccgtgtgaaatggtcccatctcagactgccctgggtagacctagactggctcatagacatctcctgccagatcacggagctcgacctttctgccaactgcctggcgaccctcccctcggttatcccctggggcctcatcaatctccggaagctgaacctctccgacaaccacctgggggagctgcctggcgtgcagtcatcggacgaaatcatctgttccaggctacttgaaattgacatttccagcaacaagttgtcccacctccctcctggattcttgcacctctcaaaacttcaaaaactgacagcttcaaaaaattgtttagaaaaattgttcgaagaagaaaatgccactaactggataggtttacggaagctacaggaacttgatatatctgacaataaattgacagaactccctgccctgttccttcactctttcaagtccctcaattctctgaatgtctccagaaacaacctgaaggtgtttccagatccctgggcctgccctttgaaatgttgtaaagcttccagaaatgccctggaatgtctgccagacaaaatggctgtcttttggaaaaatcacctgaaggatgtggatttctcagaaaacgcactcaaagaagttcccctgggacttttccagcttgatgccctcatgttcttgaggttacaggggaaccagctggcggcacttccacctcaagagaagtggacctgcaggcagctcaaaaccctggatctctccagaaaccaacttggcaaaaatgaagatggactgaaaacgaagcgtattgcctttttcaccaccagaggtcgccagcgctccgggactgaggcagagacaactatggagttcagtgcatctctggtaaccattgtgttcctgtctaacaactgtaacctctgtgcatacacatgtgcagcaagtgtgctggaatttccggccttcctaagtgagtctttggaagtcctttgcctgaacgacaaccacctcgacacagtccctccctcggtttgcctactgaagagcttatcagagctctacttgggaaacaaccctggcctccgggagctccctcctgagctggggcagctgggcaacctctggcagctggacactgaagacctgaccatcagcaatgtgcctgcagaaatccaaaaagaaggccccaaagcaatgctgtcttacctgcgtgctcagctgcggaaagcggaaaagtgcaagctgatgaagatgatcatcgtgggtcccccgcgccagggcaagtccaccctcctggagatcttacagacggggagggccccccaggtggtgcatggagaggccaccatcaggaccaccaagtgggagctccagaggccggctggctcgagagccaaggtcaaggatggtctgcgtgcagagtccctgtgggttgagtccgtggagttcaacgtctgggacatcgggggaccggccagcatggccactgtcaaccagtgcttcttcacggacaaggccctgtacgtggtggtctggaacctggcgctgggggaggaggccgtggccaacctccagttctggctgctcaacatcgaggccaaggccccaaacgccgtggtgctggtggtcgggacgcacctggatttaattgaagccaagttccgtgtggaaaggattgcaacgctgcgtgcctatgtgctggcactctgccgctccccctccggctccagggccacaggcttcccagacatcaccttcaaacacttacatgagatttcctgcaagagcctggaaggtcaggaagggctgcgacagctgattttccacgtcacgtgcagcatgaaggacgtcggcagcaccatcggctgccagcgactggcagggcggctgatccccaggagctacctgagcctgcaggaggccgtgctggcagagcagcagcgccgcagccgggacgacgacgtgcagtacctgacggacaggcagctggagcagctggtggagcagacgcccgacaacgacatcaaggactacgaggacctgcagtcagccatcagcttcctcatagaaaccggcaccctgctccatttcccggacaccagccacggcctgaggaacctctacttcctcgaccctatttggctctccgaatgtctgcagaggatctttaatattaagggctctcggtcagtggccaagaatggggtgatcagagcagaagacctcaggatgctgctggtggggactggcttcacgcagcagacggaagagcagtacttccagttcctggccaagtttgagatcgccctgcccgtcgccaatgacagctacctcctgccccatctccttccatctaaacctggcctggacacccacggtatgcggcaccccacagccaacaccattcagagggtatttaagatgagcttcgttcccgttggcttctggcaaaggtttatagcacggatgctgatcagcctggcggagatggacctgcagctttttgaaaacaagaagaatactaaaagcaggaacaggaaagtcaccatttacagttttacaggaaaccagagaaatcgctgtagcacattcagagtgaaaagaaatcagaccatctattggcaggaagggctcctggtcacttttgatgggggctacctcagtgtggaatcttccgacgtgaactggaaaaagaagaaaagcggaggaatgaaaattgtttgccaatcagaagtgagggacttctcagccatggctttcatcacggaccacgtcaattccttgattgatcagtggtttcccgccctgacagccacagagagcgacgggacgccactcatggagcagtacgtgccctgcccggtctgcgagacagcctgggcccagcacacggaccccagtgagaaatcagaggatgtgcagtacttcgacatggaagactgtgtcctgacggccatcgagcgggacttcatctcctgccccagacacccggacctccccgtgccgctgcaggagctggtccctgaactgttcatgaccgacttcccggccaggctcttcctggagaacagcaagctggagcacagcgaggacgagggcagcgtcctgggccagggcggcagtggcaccgtcatctaccgggcccggtaccagggccagcctgtggccgtcaagcgcttccacatcaaaaaattcaagaactttgctaacgtaccggcagacaccatgctgaggcacctgcgggccaccgatgccatgaagaacttctccgagttccggcaggaggccagcatgctgcacgcgctgcagcacccctgcatcgtggcgctcatcggcatcagcatccacccgctctgcttcgccctggagctcgcgccgctcagcagcctcaacaccgtgctgtccgagaacgccagagattcttcctttatacccctgggacacatgctcacccaaaaaatagcctaccagatcgcctcgggcctggcctacctgcacaagaaaaacatcatcttctgtgacctgaagtcggacaacattctggtgtggtcccttgacgtcaaggagcacatcaacatcaagctatctgactacgggatttcgaggcagtcattccatgagggcgccctaggcgtggagggcactcctggctaccaggccccagagatcaggcctcgcattgtatatgatgagaaggtagatatgttctcctatggaatggtgctctacgagttgctgtcaggacagcgccctgcactgggccaccaccagctccagattgccaagaagctgtccaagggcatccgcccggttctggggcagccggaggaagtgcagttccggcgactgcaggcgctcatgatggagtgctgggacactaagccagagaagcgaccgctggccctgtcggtggtgagccagatgaaggacccgacttttgccaccttcatgtatgaactgtgctgtgggaagcagacagccttcttctcatcccagggccaggagtacaccgtggtgttttgggatggaaaagaggagtccaggaactacacggtggtgaacacagagaagggcctcatggaggtgcagaggatgtgctgccctgggatgaaggtgagctgccagctccaggtccagagatccctgtggacagccaccgagaattcctacctggtcttagcgggcctcgccgatgggcttgtggctgtgtttcccgtggtgcggggcaccccaaaggacagctgctcctacctgtgctcacacacagccaacaggtccaagttcagcatcgcggatgaagacgcacggcagaacccctacccagtgaaggccatggaggtggtcaacagcggctctgaggtctggtacagcaatgggccgggcctccttgtcatcgactgtgcctccctggagatctgcaggcggctggagccctacatggccccctccatggttacgtcagtcgtgtgcagctctgagggcagaggggaggaggtcgtctggtgcctggatgacaaggccaactccttggtgatgtaccactccaccacctaccagctgtgtgcccggtacttctgcggggtccccagccccctcagggacatgtttcccgtgcggcccttggacacggaacccccggcagccagccacacggccaacccaaaggtgcctgagggggactccatcgcggacgtgagcatcatgtacagtgaggagctgggcacgcagatcctgatccaccaggaatcactcactgactactgctccatgtcctcctactcctcatccccaccccgccaggctgccaggtccccctcaagcctccccagctccccagcaagttcttccagtgtgcctttctccaccgactgcgaggactcagacatgctacatacgcccggtgctgcctccgacaggtctgagcatgacctgacccccatggacggggagaccttcagccagcacctgcaggccgtgaagatcctcgccgtcagagacctcatttgggtccccaggcgcggtggagatgttatcgtcattggcctggagaaggattctggcgcccagcggggccgagtcattgccgtcttaaaagcccgagagctgactccgcatggggtgctggtggatgctgccgtggtggcaaaggacactgttgtgtgcacctttgaaaatgaaaacacagagtggtgcctggccgtctggaggggctggggcgccagggagttcgacattttctaccagtcctacgaggagctgggccggctggaggcttgcactcgcaagagaaggtaattcctgtggaatgactgtcacacatcagagctggctggcccggggctgcagcctgactcctctgccatcggcctctagttctccaaggacctagaagacagatggagttctcccctgaactccttgctgctaagaagtgctgagaagttactcgcctggcggtggctccagggttctctggttctctggagcagagttctctgaataccccatcccccaactgctgattttacagccccagggaagacagtggtatcaggctgggagcggcctcctctggcctcccccatcagtttgcaggagcaggggtgcaggatcctgttctgagctgggtcaaacaaagcagggccgggccttcctgccatccccaggtctcagatggaattacactagaggccctccgctgggaagcacttgaggtagggcaggaggggggctgtgacccctgccctttccccgccagagacctcaggctctcagcacattccacaggctcctgagtccccgaggcctgggccagcttgggcaagccaagatcagatgtctctgtgttcgggaaggtctccgtgtgggaaagcccttgggggatcccgggtgaggagtgttgccccatccagagaatgaatgagttcctttaagtgccaccgccagcaagcccagaggcacacattctgagtgcacccgcttagcctttacattcctctccaccgacaaaaggaaggggaaactcaatcagcaggacttcagaaagggccttgtgtttatagctttgtcaagtaaatttggacgcagctggaaacacaggcctgtttgttgcacata

SEQ ID NO: 2 - protein, 2013 aa, Homo sapiens
METLNGAGDTGGKPSTRGGDPAARSRRTEGIRAAYRRGDRGGARDLLEEACDQCASQLEKGQLLSIPAAYGDLEMVRYLLSKRLVELPTEPTDDNPAVVAAYFGHTAVVQNTLPTEPTDDNPAVVAAYFGHTAVVQELLESLPGPCSPQRLLNWMLALACQRGHLGVVKLLVLTHGADPESYAVRKNEFPVIVRLPLYAAIKSGNEDIAIFLLRHGAYFCSYILLDSPDPSKHLLRKYFIEASPLPSSYPGKTALRVKWSHLRLPWVDLDWLIDISCQITELDLSANCLATLPSVIPWGLINLRKLNLSDNHLGELPGVQSSDEIICSRLLEIDISSNKLSHLPPGFLHLSKLQKLTASKNCLEKLFEEENATNWIGLRKLQELDISDNKLTELPALFLHSFKSLNSLNVSRNNLKVFPDPWACPLKCCKASRNALECLPDKMAVFWKNHLKDVDFSENALKEVPLGLFQLDALMFLRLQGNQLAALPPQEKWTCRQLKTLDLSRNQLGKNEDGLKTKRIAFFTTRGRQRSGTEAETTMEFSASLVTIVFLSNNCNLCAYTCAASVLEFPAFLSESLEVLCLNDNHLDTVPPSVCLLKSLSELYLGNNPGLRELPPELGQLGNLWQLDTEDLTISNVPAEIQKEGPKAMLSYLRAQLRKAEKCKLMKMIIVGPPRQGKSTLLEILQTGRAPQVVHGEATIRTTKWELQRPAGSRAKVKDGLRAESLWVESVEFNVWDIGGPASMATVNQCFFTDKALYVVVWNLALGEEAVANLQFWLLNIEAKAPNAVVLVVGTHLDLIEAKFRVERIATLRAYVLALCRSPSGSRATGFPDITFKHLHEISCKSLEGQEGLRQLIFHVTCSMKDVGSTIGCQRLAGRLIPRSYLSLQEAVLAEQQRRSRDDDVQYLTDRQLEQLVEQTPDNDIKDYEDLQSAISFLIETGTLLHFPDTSHGLRNLYFLDPIWLSECLQRIFNIKGSRSVAKNGVIRAEDLRMLLVGTGFTQQTEEQYFQFLAKFEIALPVANDSYLLPHLLPSKPGLDTHGMRHPTANTIQRVFKMSFVPVGFWQRFIARMLISLAEMDLQLFENKKNTKSRNRKVTIYSFTGNQRNRCSTFRVKRNQTIYWQEGLLVTFDGGYLSVESSDVNWKKKKSGGMKIVCQSEVRDFSAMAFITDHVNSLIDQWFPALTATESDGTPLMEQYVPCPVCETAWAQHTDPSEKSEDVQYFDMEDCVLTAIERDFISCPRHPDLPVPLQELVPELFMTDFPARLFLENSKLEHSEDEGSVLGQGGSGTVIYRARYQGQPVAVKRFHIKKFKNFANVPADTMLRHLRATDAMKNFSEFRQEASMLHALQHPCIVALIGISIHPLCFALELAPLSSLNTVLSENARDSSFIPLGHMLTQKIAYQIASGLAYLHKKNIIFCDLKSDNILVWSLDVKEHINIKLSDYGISRQSFHEGALGVEGTPGYQAPEIRPRIVYDEKVDMFSYGMVLYELLSGQRPALGHHQLQIAKKLSKGIRPVLGQPEEVQFRRLQALMMECWDTKPEKRPLALSVVSQMKDPTFATFMYELCCGKQTAFFSSQGQEYTVVFWDGKEESRNYTVVNTEKGLMEVQRMCCPGMKVSCQLQVQRSLWTATENSYLVLAGLADGLVAVFPVVRGTPKDSCSYLCSHTANRSKFSIADEDARQNPYPVKAMEVVNSGSEVWYSNGPGLLVIDCASLEICRRLEPYMAPSMVTSVVCSSEGRGEEVVWCLDDKANSLVMYHSTTYQLCARYFCGVPSPLRDMFPVRPLDTEPPAASHTANPKVPEGDSIADVSIMYSEELGTQILIHQESLTDYCSMSSYSSSPPRQAARSPSSLPSSPASSSSVPFSTDCEDSDMLHTPGAASDRSEHDLTPMDGETFSQHLQAVKILAVRDLIWVPRRGGDVIVIGLEKDSGAQRGRVIAVLKARELTPHGVLVDAAVVAKDTVVCTFENENTEWCLAVWRGWGAREFDIFYQSYEELGRLEACTRKR

SEQ ID NO: 3 - DNA, 7307 bp, Homo sapiens
gcggccgccgaggggacgcctcgtgacggttcctgggagagctggcggcggccttgctctgcgcgctcttcgcgccgccctccccgcccgcccgcctcaggattgaggaagtgcgtctgggcccggccccggcgcggggggcagacggcggtgggacggccaggccccggccccgccagtgtgtccgcccggccccgcgtcccggagcgcccgcacccggccccgccgccgcctcagagcaagaaagctttctgctcagccatggctacgagtccacgccttaatgcaccccacagccagcggcagtggcagtgacaacagcgggacctgcctttgaagatcggctgctgcaagggttgatggctggcatgtcgcaaagaccccccagcatgtactggtgtgtggggccggaggagtcagctgtgtgtccagaacgtgccatggagacgcttaacggtgccggggacacgggcggcaagccgtccacgcggggcggtgaccctgcagcgcggtcccgcaggacggaaggcatccgcgccgcgtacaggcggggagaccgcggcggcgcccgggacctgctggaggaggcctgcgaccagtgcgcgtcccagctggaaaagggccagcttctgagcatcccggcagcctatggggatctggagatggtccgctacctactcagcaagagactggtggagctgcccaccgagcccacggatgacaacccagccgtggtggcagcgtattttggacacacggcagttgtgcaaaatacgctgcccaccgagcccacggatgacaacccagccgtggtggcagcgtattttggacacacggcagttgtgcaggaattgcttgagtccttaccaggtccctgcagtccccagcggcttctgaactggatgctggccttggcttgccagcgagggcacctgggggttgtgaagctcctggtcctgacgcacggggctgacccggagagctacgctgtcaggaagaatgagttccctgtcatcgtgcgcttgcccctgtatgcggccatcaagtcagggaatgaagacattgcaatattcctgcttcggcatggggcctatttctgttcctacatcttgctggatagtcctgaccccagcaaacatctgctgagaaagtacttcattgaagccagtcccttgcccagcagttatccgggaaaaacagctctccgtgtgaaatggtcccatctcagactgccctgggtagacctagactggctcatagacatctcctgccagatcacggagctcgacctttctgccaactgcctggcgaccctcccctcggttatcccctggggcctcatcaatctccggaagctgaacctctccgacaaccacctgggggagctgcctggcgtgcagtcatcggacgaaatcatctgttccaggctacttgaaattgacatttccagcaacaagttgtcccacctccctcctggattcttgcacctctcaaaacttcaaaaactgacagcttcaaaaaattgtttagaaaaattgttcgaagaagaaaatgccactaactggataggtttacggaagctacaggaacttgatatatctgacaataaattgacagaactccctgccctgttccttcactctttcaagtccctcaattctctgaatgtctccagaaacaacctgaaggtgtttccagatccctgggcctgccctttgaaatgttgtaaagcttccagaaatgccctggaatgtctgccagacaaaatggctgtcttttggaaaaatcacctgaaggatgtggatttctcagaaaacgcactcaaagaagttcccctgggacttttccagcttgatgccctcatgttcttgaggttacaggggaaccagctggcggcacttccacctcaagagaagtggacctgcaggcagctcaaaaccctggatctctccagaaaccaacttggcaaaaatgaagatggactgaaaacgaagcgtattgcctttttcaccaccagaggtcgccagcgctccgggactgaggcagagacaactatggagttcagtgcatctctggtaaccattgtgttcctgtctaacaactgtaacctctgtgcatacacatgtgcagcaagtgtgctggaatttccggccttcctaagtgagtctttggaagtcctttgcctgaacgacaaccacctcgacacagtccctccctcggtttgcctactgaagagcttatcagagctctacttgggaaacaaccctggcctccgggagctccctcctgagctggggcagctgggcaacctctggcagctggacactgaagacctgaccatcagcaatgtgcctgcagaaatccaaaaagaaggccccaaagcaatgctgtcttacctgcgtgctcagctgcggaaagcggaaaagtgcaagctgatgaagatgatcatcgtgggtcccccgcgccagggcaagtccaccctcctggagatcttacagacggggagggccccccaggtggtgcatggagaggccaccatcaggaccaccaagtgggagctccagaggccggctggctcgagagccaaggtcaaggatggtctgcgtgcagagtccctgtgggttgagtccgtggagttcaacgtctgggacatcgggggaccggccagcatggccactgtcaaccagtgcttcttcacggacaaggccctgtacgtggtggtctggaacctggcgctgggggaggaggccgtggccaacctccagttctggctgctcaacatcgaggccaaggccccaaacgccgtggtgctggtggtcgggacgcacctggatttaattgaagccaagttccgtgtggaaaggattgcaacgctgcgtgcctatgtgctggcactctgccgctccccctccggctccagggccacaggcttcccagacatcaccttcaaacacttacatgagatttcctgcaagagcctggaaggtcaggaagggctgcgacagctgattttccacgtcacgtgcagcatgaaggacgtcggcagcaccatcggctgccagcgactggcagggcggctgatccccaggagctacctgagcctgcaggaggccgtgctggcagagcagcagcgccgcagccgggacgacgacgtgcagtacctgacggacaggcagctggagcagctggtggagcagacgcccgacaacgacatcaaggactacgaggacctgcagtcagccatcagcttcctcatagaaaccggcaccctgctccatttcccggacaccagccacggcctgaggaacctctacttcctcgaccctatttggctctccgaatgtctgcagaggatctttaatattaagggctctcggtcagtggccaagaatggggtgatcagagcagaagacctcaggatgctgctggtggggactggcttcacgcagcagacggaagagcagtacttccagttcctggccaagtttgagatcgccctgcccgtcgccaatgacagctacctcctgccccatctccttccatctaaacctggcctggacacccacggtatgcggcaccccacagccaacaccattcagagggtatttaagatgagcttcgttcccgttggcttctggcaaaggtttatagcacggatgctgatcagcctggcggagatggacctgcagctttttgaaaacaagaagaatactaaaagcaggaacaggaaagtcaccatttacagttttacaggaaaccagagaaatcgctgtagcacattcagagtgaaaagaaatcagaccatctattggcaggaagggctcctggtcacttttgatgggggctacctcagtgtggaatcttccgacgtgaactggaaaaagaagaaaagcggaggaatgaaaattgtttgccaatcagaagtgagggacttctcagccatggctttcatcacggaccacgtcaattccttgattgatcagtggtttcccgccctgacagccacagagagcgacgggacgccactcatggagcagtacgtgccctgcccggtctgcgagacagcctgggcccagcacacggaccccagtgagaaatcagaggatgtgcagtacttcgacatggaagactgtgtcctgacggccatcgagcgggacttcatctcctgccccagacacccggacctccccgtgccgctgcaggagctggtccctgaactgttcatgaccgacttcccggccaggctcttcctggagaacagcaagctggagcacagcgaggacgagggcagcgtcctgggccagggcggcagtggcaccgtcatctaccgggcccggtaccagggccagcctgtggccgtcaagcgcttccacatcaaaaaattcaagaactttgctaacgtaccggcagacaccatgctgaggcacctgcgggccaccgatgccatgaagaacttctccgagttccggcaggaggccagcatgctgcacgcgctgcagcacccctgcatcgtggcgctcatcggcatcagcatccacccgctctgcttcgccctggagctcgcgccgctcagcagcctcaacaccgtgctgtccgagaacgccagagattcttcctttatacccctgggacacatgctcacccaaaaaatagcctaccagatcgcctcgggcctggcctacctgcacaagaaaaacatcatcttctgtgacctgaagtcggacaacattctggtgtggtcccttgacgtcaaggagcacatcaacatcaagctatctgactacgggatttcgaggcagtcattccatgagggcgccctaggcgtggagggcactcctggctaccaggccccagagatcaggcctcgcattgtatatgatgagaaggtagatatgttctcctatggaatggtgctctacgagttgctgtcaggacagcgccctgcactgggccaccaccagctccagattgccaagaagctgtccaagggcatccgcccggttctggggcagccggaggaagtgcagttccggcgactgcaggcgctcatgatggagtgctgggacactaagccagagaagcgaccgctggccctgtcggtggtgagccagatgaaggacccgacttttgccaccttcatgtatgaactgtgctgtgggaagcagacagccttcttctcatcccagggccaggagtacaccgtggtgttttgggatggaaaagaggagtccaggaactacacggtggtgaacacagagaagggcctcatggaggtgcagaggatgtgctgccctgggatgaaggtgagctgccagctccaggtccagagatccctgtggacagccaccgagaattcctacctggtcttagcgggcctcgccgatgggcttgtggctgtgtttcccgtggtgcggggcaccccaaaggacagctgctcctacctgtgctcacacacagccaacaggtccaagttcagcatcgcggatgaagacgcacggcagaacccctacccagtgaaggccatggaggtggtcaacagcggctctgaggtctggtacagcaatgggccgggcctccttgtcatcgactgtgcctccctggagatctgcaggcggctggagccctacatggccccctccatggttacgtcagtcgtgtgcagctctgagggcagaggggaggaggtcgtctggtgcctggatgacaaggccaactccttggtgatgtaccactccaccacctaccagctgtgtgcccggtacttctgcggggtccccagccccctcagggacatgtttcccgtgcggcccttggacacggaacccccggcagccagccacacggccaacccaaaggtgcctgagggggactccatcgcggacgtgagcatcatgtacagtgaggagctgggcacgcagatcctgatccaccaggaatcactcactgactactgctccatgtcctcctactcctcatccccaccccgccaggctgccaggtccccctcaagcctccccagctccccagcaagttcttccagtgtgcctttctccaccgactgcgaggactcagacatgctacatacgcccggtgctgcctccgacaggtctgagcatgacctgacccccatggacggggagaccttcagccagcacctgcaggccgtgaagatcctcgccgtcagagacctcatttgggtccccaggcgcggtggagatgttatcgtcattggcctggagaaggattctggcgcccagcggggccgagtcattgccgtcttaaaagcccgagagctgactccgcatggggtgctggtggatgctgccgtggtggcaaaggacactgttgtgtgcacctttgaaaatgaaaacacagagtggtgcctggccgtctggaggggctggggcgccagggagttcgacattttctaccagtcctacgaggagctgggccggctggaggcttgcactcgcaagagaaggtaattcctgtggaatgactgtcacacatcagagctggctggcccggggctgcagcctgactcctctgccatcggcctctagttctccaaggacctagaagacagatggagttctcccctgaactccttgctgctaagaagtgctgagaagttactcgcctggcggtggctccagggttctctggttctctggagcagagttctctgaataccccatcccccaactgctgattttacagccccagggaagacagtggtatcaggctgggagcggcctcctctggcctcccccatcagtttgcaggagcaggggtgcaggatcctgttctgagctgggtcaaacaaagcagggccgggccttcctgccatccccaggtctcagatggaattacactagaggccctccgctgggaagcacttgaggtagggcaggaggggggctgtgacccctgccctttccccgccagagacctcaggctctcagcacattccacaggctcctgagtccccgaggcctgggccagcttgggcaagccaagatcagatgtctctgtgttcgggaaggtctccgtgtgggaaagcccttgggggatcccgggtgaggagtgttgccccatccagagaatgaatgagttcctttaagtgccaccgccagcaagcccagaggcacacattctgagtgcacccgcttagcctttacattcctctccaccgacaaaaggaaggggaaactcaatcagcaggacttcagaaagggccttgtgtttatagctttgtcaagtaaatttggacgcagctggaaacacaggcctgtttgttgcacata

SEQ ID NO: 4 - protein, 2040 aa, Homo sapiens
MAGMSQRPPSMYWCVGPEESAVCPERAMETLNGAGDTGGKPSTRGGDPAARSRRTEGIRAAYRRGDRGGARDLLEEACDQCASQLEKGQLLSIPAAYGDLEMVRYLLSKRLVELPTEPTDDNPAVVAAYFGHTAVVQNTLPTEPTDDNPAVVAAYFGHTAVVQELLESLPGPCSPQRLLNWMLALACQRGHLGVVKLLVLTHGADPESYAVRKNEFPVIVRLPLYAAIKSGNEDIAIFLLRHGAYFCSYILLDSPDPSKHLLRKYFIEASPLPSSYPGKTALRVKWSHLRLPWVDLDWLIDISCQITELDLSANCLATLPSVIPWGLINLRKLNLSDNHLGELPGVQSSDEIICSRLLEIDISSNKLSHLPPGFLHLSKLQKLTASKNCLEKLFEEENATNWIGLRKLQELDISDNKLTELPALFLHSFKSLNSLNVSRNNLKVFPDPWACPLKCCKASRNALECLPDKMAVFWKNHLKDVDFSENALKEVPLGLFQLDALMFLRLQGNQLAALPPQEKWTCRQLKTLDLSRNQLGKNEDGLKTKRIAFFTTRGRQRSGTEAETTMEFSASLVTIVFLSNNCNLCAYTCAASVLEFPAFLSESLEVLCLNDNHLDTVPPSVCLLKSLSELYLGNNPGLRELPPELGQLGNLWQLDTEDLTISNVPAEIQKEGPKAMLSYLRAQLRKAEKCKLMKMIIVGPPRQGKSTLLEILQTGRAPQVVHGEATIRTTKWELQRPAGSRAKVKDGLRAESLWVESVEFNVWDIGGPASMATVNQCFFTDKALYVVVWNLALGEEAVANLQFWLLNIEAKAPNAVVLVVGTHLDLIEAKFRVERIATLRAYVLALCRSPSGSRATGFPDITFKHLHEISCKSLEGQEGLRQLIFHVTCSMKDVGSTIGCQRLAGRLIPRSYLSLQEAVLAEQQRRSRDDDVQYLTDRQLEQLVEQTPDNDIKDYEDLQSAISFLIETGTLLHFPDTSHGLRNLYFLDPIWLSECLQRIFNIKGSRSVAKNGVIRAEDLRMLLVGTGFTQQTEEQYFQFLAKFEIALPVANDSYLLPHLLPSKPGLDTHGMRHPTANTIQRVFKMSFVPVGFWQRFIARMLISLAEMDLQLFENKKNTKSRNRKVTIYSFTGNQRNRCSTFRVKRNQTIYWQEGLLVTFDGGYLSVESSDVNWKKKKSGGMKIVCQSEVRDFSAMAFITDHVNSLIDQWFPALTATESDGTPLMEQYVPCPVCETAWAQHTDPSEKSEDVQYFDMEDCVLTAIERDFISCPRHPDLPVPLQELVPELFMTDFPARLFLENSKLEHSEDEGSVLGQGGSGTVIYRARYQGQPVAVKRFHIKKFKNFANVPADTMLRHLRATDAMKNFSEFRQEASMLHALQHPCIVALIGISIHPLCFALELAPLSSLNTVLSENARDSSFIPLGHMLTQKIAYQIASGLAYLHKKNIIFCDLKSDNILVWSLDVKEHINIKLSDYGISRQSFHEGALGVEGTPGYQAPEIRPRIVYDEKVDMFSYGMVLYELLSGQRPALGHHQLQIAKKLSKGIRPVLGQPEEVQFRRLQALMMECWDTKPEKRPLALSVVSQMKDPTFATFMYELCCGKQTAFFSSQGQEYTVVFWDGKEESRNYTVVNTEKGLMEVQRMCCPGMKVSCQLQVQRSLWTATENSYLVLAGLADGLVAVFPVVRGTPKDSCSYLCSHTANRSKFSIADEDARQNPYPVKAMEVVNSGSEVWYSNGPGLLVIDCASLEICRRLEPYMAPSMVTSVVCSSEGRGEEVVWCLDDKANSLVMYHSTTYQLCARYFCGVPSPLRDMFPVRPLDTEPPAASHTANPKVPEGDSIADVSIMYSEELGTQILIHQESLTDYCSMSSYSSSPPRQAARSPSSLPSSPASSSSVPFSTDCEDSDMLHTPGAASDRSEHDLTPMDGETFSQHLQAVKILAVRDLIWVPRRGGDVIVIGLEKDSGAQRGRVIAVLKARELTPHGVLVDAAVVAKDTVVCTFENENTEWCLAVWRGWGAREFDIFYQSYEELGRLEACTRKR

SEQ ID NO: 5 - DNA, 7015 bp, Homo sapiens
cgaggggacgcctcgcgacggttcctgggagagctggcggcggccttgctctgcgcgctcttcgcgccgccctccccgcccgcccgcctcaggattgaggaagtgcgtctgggcccggccccggcgcggggggcagacggcggtgggacggccaggccccggccccgccagtgtgtccgcccggccccgcgtcccggaggagtcagctgtgtgtccagaacgtgccatggagacgcttaacggtgccggggacacgggcggcaagccgtccacgcggggcggtgaccctgcagcgcggtcccgcaggacggaaggcatccgcgccgcgtacaggcggggagaccgcggcggcgcccgggacctgctggaggaggcctgcgaccagtgcgcgtcccagctggaaaagggccagcttctgagcatcccggcagcctatggggatctggagatggtccgctacctactcagcaagagactggtggagctgcccaccgagcccacggatgacaacccagccgtggtggcagcgtattttggacacacggcagttgtgcaggaattgcttgagtccttaccaggtccctgcagtccccagcggcttctgaactggatgctggccttggcttgccagcgagggcacctgggggttgtgaagctcctggtcctgacgcacggggctgacccggagagctacgctgtcaggaagaatgagttccctgtcatcgtgcgcttgcccctgtatgcggccatcaagtcagggaatgaagacattgcaatattcctgcttcggcatggggcctatttctgttcctacatcttgctggatagtcctgaccccagcaaacatctgctgagaaagtacttcattgaagccagtcccttgcccagcagttatccgggaaaaacagctctccgtgtgaaatggtcccatctcagactgccctgggtagacctagactggctcatagacatctcctgccagatcacggagctcgacctttctgccaactgcctggcgaccctcccctcggttatcccctggggcctcatcaatctccggaagctgaacctctccgacaaccacctgggggagctgcctggcgtgcagtcatcggacgaaatcatctgttccaggctacttgaaattgacatttccagcaacaagttgtcccacctccctcctggattcttgcacctctcaaaacttcaaaaactgacagcttcaaaaaattgtttagaaaaattgttcgaagaagaaaatgccactaactggataggtttacggaagctacaggaacttgatatatctgacaataaattgacagaactccctgccctgttccttcactctttcaagtccctcaattctctgaatgtctccagaaacaacctgaaggtgtttccagatccctgggcctgccctttgaaatgttgtaaagcttccagaaatgccctggaatgtctgccagacaaaatggctgtcttttggaaaaatcacctgaaggatgtggatttctcagaaaacgcactcaaagaagttcccctgggacttttccagcttgatgccctcatgttcttgaggttacaggggaaccagctggcggcacttccacctcaagagaagtggacctgcaggcagctcaaaaccctggatctctccagaaaccaacttggcaaaaatgaagatggactgaaaacgaagcgtattgcctttttcaccaccagaggtcgccagcgctccgggactgaggcagagacaactatggagttcagtgcatctctggtaaccattgtgttcctgtctaacaactgtaacctctgtgcatacacatgtgcagcaagtgtgctggaatttccggccttcctaagtgagtctttggaagtcctttgcctgaacgacaaccacctcgacacagtccctccctcggtttgcctactgaagagcttatcagagctctacttgggaaacaaccctggcctccgggagctccctcctgagctggggcagctgggcaacctctggcagctggacactgaagacctgaccatcagcaatgtgcctgcagaaatccaaaaagaaggccccaaagcaatgctgtcttacctgcgtgctcagctgcggaaagcggaaaagtgcaagctgatgaagatgatcatcgtgggtcccccgcgccagggcaagtccaccctcctggagatcttacagacggggagggccccccaggtggtgcatggagaggccaccatcaggaccaccaagtgggagctccagaggccggctggctcgagagccaaggtcaaggatggtctgcgtgcagagtccctgtgggttgagtccgtggagttcaacgtctgggacatcgggggaccggccagcatggccactgtcaaccagtgcttcttcacggacaaggccctgtacgtggtggtctggaacctggcgctgggggaggaggccgtggccaacctccagttctggctgctcaacatcgaggccaaggccccaaacgccgtggtgctggtggtcgggacgcacctggatttaattgaagccaagttccgtgtggaaaggattgcaacgctgcgtgcctatgtgctggcactctgccgctccccctccggctccagggccacaggcttcccagacatcaccttcaaacacttacatgagatttcctgcaagagcctggaaggtcaggaagggctgcgacagctgattttccacgtcacgtgcagcatgaaggacgtcggcagcaccatcggctgccagcgactggcagggcggctgatccccaggagctacctgagcctgcaggaggccgtgctggcagagcagcagcgccgcagccgggacgacgacgtgcagtacctgacggacaggcagctggagcagctggtggagcagacgcccgacaacgacatcaaggactacgaggacctgcagtcagccatcagcttcctcatagaaaccggcaccctgctccatttcccggacaccagccacggcctgaggaacctctacttcctcgaccctatttggctctccgaatgtctgcagaggatctttaatattaagggctctcggtcagtggccaagaatggggtgatcagagcagaagacctcaggatgctgctggtggggactggcttcacgcagcagacggaagagcagtacttccagttcctggccaagtttgagatcgccctgcccgtcgccaatgacagctacctcctgccccatctccttccatctaaacctggcctggacacccacggtatgcggcaccccacagccaacaccattcagagggtatttaagatgagcttcgttcccgttggcttctggcaaaggtttatagcacggatgctgatcagcctggcggagatggacctgcagctttttgaaaacaagaagaatactaaaagcaggaacaggaaagtcaccatttacagttttacaggaaaccagagaaatcgctgtagcacattcagagtgaaaagaaatcagaccatctattggcaggaagggctcctggtcacttttgatgggggctacctcagtgtggaatcttccgacgtgaactggaaaaagaagaaaagcggaggaatgaaaattgtttgccaatcagaagtgagggacttctcagccatggctttcatcacggaccacgtcaattccttgattgatcagtggtttcccgccctgacagccacagagagcgacgggacgccactcatggagcagtacgtgccctgcccggtctgcgagacagcctgggcccagcacacggaccccagtgagaaatcagaggatgtgcagtacttcgacatggaagactgtgtcctgacggccatcgagcgggacttcatctcctgccccagacacccggacctccccgtgccgctgcaggagctggtccctgaactgttcatgaccgacttcccggccaggctcttcctggagaacagcaagctggagcacagcgaggacgagggcagcgtcctgggccagggcggcagtggcaccgtcatctaccgggcccggtaccagggccagcctgtggccgtcaagcgcttccacatcaaaaaattcaagaactttgctaacgtaccggcagacaccatgctgaggcacctgcgggccaccgatgccatgaagaacttctccgagttccggcaggaggccagcatgctgcacgcgctgcagcacccctgcatcgtggcgctcatcggcatcagcatccacccgctctgcttcgccctggagctcgcgccgctcagcagcctcaacaccgtgctgtccgagaacgccagagattcttcctttatacccctgggacacatgctcacccaaaaaatagcctaccagatcgcctcgggcctggcctacctgcacaagaaaaacatcatcttctgtgacctgaagtcggacaacattctggtgtggtcccttgacgtcaaggagcacatcaacatcaagctatctgactacgggatttcgaggcagtcattccatgagggcgccctaggcgtggagggcactcctggctaccaggccccagagatcaggcctcgcattgtatatgatgagaaggtagatatgttctcctatggaatggtgctctacgagttgctgtcaggacagcgccctgcactgggccaccaccagctccagattgccaagaagctgtccaagggcatccgcccggttctggggcagccggaggaagtgcagttccggcgactgcaggcgctcatgatggagtgctgggacactaagccagagaagcgaccgctggccctgtcggtggtgagccagatgaaggacccgacttttgccaccttcatgtatgaactgtgctgtgggaagcagacagccttcttctcatcccagggccaggagtacaccgtggtgttttgggatggaaaagaggagtccaggaactacacggtggtgaacacagagaagggcctcatggaggtgcagaggatgtgctgccctgggatgaaggtgagctgccagctccaggtccagagatccctgtggacagccaccgagaattcctacctggtcttagcgggcctcgccgatgggcttgtggctgtgtttcccgtggtgcggggcaccccaaaggacagctgctcctacctgtgctcacacacagccaacaggtccaagttcagcatcgcggatgaagacgcacggcagaacccctacccagtgaaggccatggaggtggtcaacagcggctctgaggtctggtacagcaatgggccgggcctccttgtcatcgactgtgcctccctggagatctgcaggcggctggagccctacatggccccctccatggttacgtcagtcgtgtgcagctctgagggcagaggggaggaggtcgtctggtgcctggatgacaaggccaactccttggtgatgtaccactccaccacctaccagctgtgtgcccggtacttctgcggggtccccagccccctcagggacatgtttcccgtgcggcccttggacacggaacccccggcagccagccacacggccaacccaaaggtgcctgagggggactccatcgcggacgtgagcatcatgtacagtgaggagctgggcacgcagatcctgatccaccaggaatcactcactgactactgctccatgtcctcctactcctcatccccaccccgccaggctgccaggtccccctcaagcctccccagctccccagcaagttcttccagtgtgcctttctccaccgactgcgaggactcagacatgctacatacgcccggtgctgcctccgacaggtctgagcatgacctgacccccatggacggggagaccttcagccagcacctgcaggccgtgaagatcctcgccgtcagagacctcatttgggtccccaggcgcggtggagatgttatcgtcattggcctggagaaggattctggcgcccagcggggccgagtcattgccgtcttaaaagcccgagagctgactccgcatggggtgctggtggatgctgccgtggtggcaaaggacactgttgtgtgcacctttgaaaatgaaaacacagagtggtgcctggccgtctggaggggctggggcgccagggagttcgacattttctaccagtcctacgaggagctgggccggctggaggcttgcactcgcaagagaaggtaattcctgtggaatgactgtcacacatcagagctggctggcccggggctgcagcctgactcctctgccatcggcctctagttctccaaggacctagaagacagatggagttctcccctgaactccttgctgctaagaagtgctgagaagttactcgcctggcggtggctccagggttctctggttctctggagcagagttctctgaataccccatcccccaactgctgattttacagccccagggaagacagtggtatcaggctgggagcggcctcctctggcctcccccatcagtttgcaggagcaggggtgcaggatcctgttctgagctgggtcaaacaaagcagggccgggccttcctgccatccccaggtctcagatggaattacactagaggccctccgctgggaagcacttgaggtagggcaggaggggggctgtgacccctgccctttccccgccagagacctcaggctctcagcacattccacaggctcctgagtccccgaggcctgggccagcttgggcaagccaagatcagatgtctctgtgttcgggaaggtctccgtgtgggaaagcccttgggggatcccgggtgaggagtgttgccccatccagagaatgaatgagttcctttaagtgccaccgccagcaagcccagaggcacacattctgagtgcacccgcttagcctttacattcctctccaccgacaaaaggaaggggaaactcaatcagcaggacttcagaaagggccttgtgtttatagctttgtcaagtaaatttggacgcagctggaaacacaggcctgtttgttgcacata

SEQ ID NO: 6 - protein, 1987 aa, Homo sapiens
METLNGAGDTGGKPSTRGGDPAARSRRTEGIRAAYRRGDRGGARDLLEEACDQCASQLEKGQLLSIPAAYGDLEMVRYLLSKRLVELPTEPTDDNPAVVAAYFGHTAVVQELLESLPGPCSPQRLLNWMLALACQRGHLGVVKLLVLTHGADPESYAVRKNEFPVIVRLPLYAAIKSGNEDIAIFLLRHGAYFCSYILLDSPDPSKHLLRKYFIEASPLPSSYPGKTALRVKWSHLRLPWVDLDWLIDISCQITELDLSANCLATLPSVIPWGLINLRKLNLSDNHLGELPGVQSSDEIICSRLLEIDISSNKLSHLPPGFLHLSKLQKLTASKNCLEKLFEEENATNWIGLRKLQELDISDNKLTELPALFLHSFKSLNSLNVSRNNLKVFPDPWACPLKCCKASRNALECLPDKMAVFWKNHLKDVDFSENALKEVPLGLFQLDALMFLRLQGNQLAALPPQEKWTCRQLKTLDLSRNQLGKNEDGLKTKRIAFFTTRGRQRSGTEAETTMEFSASLVTIVFLSNNCNLCAYTCAASVLEFPAFLSESLEVLCLNDNHLDTVPPSVCLLKSLSELYLGNNPGLRELPPELGQLGNLWQLDTEDLTISNVPAEIQKEGPKAMLSYLRAQLRKAEKCKLMKMIIVGPPRQGKSTLLEILQTGRAPQVVHGEATIRTTKWELQRPAGSRAKVKDGLRAESLWVESVEFNVWDIGGPASMATVNQCFFTDKALYVVVWNLALGEEAVANLQFWLLNIEAKAPNAVVLVVGTHLDLIEAKFRVERIATLRAYVLALCRSPSGSRATGFPDITFKHLHEISCKSLEGQEGLRQLIFHVTCSMKDVGSTIGCQRLAGRLIPRSYLSLQEAVLAEQQRRSRDDDVQYLTDRQLEQLVEQTPDNDIKDYEDLQSAISFLIETGTLLHFPDTSHGLRNLYFLDPIWLSECLQRIFNIKGSRSVAKNGVIRAEDLRMLLVGTGFTQQTEEQYFQFLAKFEIALPVANDSYLLPHLLPSKPGLDTHGMRHPTANTIQRVFKMSFVPVGFWQRFIARMLISLAEMDLQLFENKKNTKSRNRKVTIYSFTGNQRNRCSTFRVKRNQTIYWQEGLLVTFDGGYLSVESSDVNWKKKKSGGMKIVCQSEVRDFSAMAFITDHVNSLIDQWFPALTATESDGTPLMEQYVPCPVCETAWAQHTDPSEKSEDVQYFDMEDCVLTAIERDFISCPRHPDLPVPLQELVPELFMTDFPARLFLENSKLEHSEDEGSVLGQGGSGTVIYRARYQGQPVAVKRFHIKKFKNFANVPADTMLRHLRATDAMKNFSEFRQEASMLHALQHPCIVALIGISIHPLCFALELAPLSSLNTVLSENARDSSFIPLGHMLTQKIAYQIASGLAYLHKKNIIFCDLKSDNILVWSLDVKEHINIKLSDYGISRQSFHEGALGVEGTPGYQAPEIRPRIVYDEKVDMFSYGMVLYELLSGQRPALGHHQLQIAKKLSKGIRPVLGQPEEVQFRRLQALMMECWDTKPEKRPLALSVVSQMKDPTFATFMYELCCGKQTAFFSSQGQEYTVVFWDGKEESRNYTVVNTEKGLMEVQRMCCPGMKVSCQLQVQRSLWTATENSYLVLAGLADGLVAVFPVVRGTPKDSCSYLCSHTANRSKFSIADEDARQNPYPVKAMEVVNSGSEVWYSNGPGLLVIDCASLEICRRLEPYMAPSMVTSVVCSSEGRGEEVVWCLDDKANSLVMYHSTTYQLCARYFCGVPSPLRDMFPVRPLDTEPPAASHTANPKVPEGDSIADVSIMYSEELGTQILIHQESLTDYCSMSSYSSSPPRQAARSPSSLPSSPASSSSVPFSTDCEDSDMLHTPGAASDRSEHDLTPMDGETFSQHLQAVKILAVRDLIWVPRRGGDVIVIGLEKDSGAQRGRVIAVLKARELTPHGVLVDAAVVAKDTVVCTFENENTEWCLAVWRGWGAREFDIFYQSYEELGRLEACTRKR

SEQ ID NO: 7 - DNA, 7229 bp, Homo sapiens
gcggccgccgaggggacgcctcgtgacggttcctgggagagctggcggcggccttgctctgcgcgctcttcgcgccgccctccccgcccgcccgcctcaggattgaggaagtgcgtctgggcccggccccggcgcggggggcagacggcggtgggacggccaggccccggccccgccagtgtgtccgcccggccccgcgtcccggagcgcccgcacccggccccgccgccgcctcagagcaagaaagctttctgctcagccatggctacgagtccacgccttaatgcaccccacagccagcggcagtggcagtgacaacagcgggacctgcctttgaagatcggctgctgcaagggttgatggctggcatgtcgcaaagaccccccagcatgtactggtgtgtggggccggaggagtcagctgtgtgtccagaacgtgccatggagacgcttaacggtgccggggacacgggcggcaagccgtccacgcggggcggtgaccctgcagcgcggtcccgcaggacggaaggcatccgcgccgcgtacaggcggggagaccgcggcggcgcccgggacctgctggaggaggcctgcgaccagtgcgcgtcccagctggaaaagggccagcttctgagcatcccggcagcctatggggatctggagatggtccgctacctactcagcaagagactggtggagctgcccaccgagcccacggatgacaacccagccgtggtggcagcgtattttggacacacggcagttgtgcaggaattgcttgagtccttaccaggtccctgcagtccccagcggcttctgaactggatgctggccttggcttgccagcgagggcacctgggggttgtgaagctcctggtcctgacgcacggggctgacccggagagctacgctgtcaggaagaatgagttccctgtcatcgtgcgcttgcccctgtatgcggccatcaagtcagggaatgaagacattgcaatattcctgcttcggcatggggcctatttctgttcctacatcttgctggatagtcctgaccccagcaaacatctgctgagaaagtacttcattgaagccagtcccttgcccagcagttatccgggaaaaacagctctccgtgtgaaatggtcccatctcagactgccctgggtagacctagactggctcatagacatctcctgccagatcacggagctcgacctttctgccaactgcctggcgaccctcccctcggttatcccctggggcctcatcaatctccggaagctgaacctctccgacaaccacctgggggagctgcctggcgtgcagtcatcggacgaaatcatctgttccaggctacttgaaattgacatttccagcaacaagttgtcccacctccctcctggattcttgcacctctcaaaacttcaaaaactgacagcttcaaaaaattgtttagaaaaattgttcgaagaagaaaatgccactaactggataggtttacggaagctacaggaacttgatatatctgacaataaattgacagaactccctgccctgttccttcactctttcaagtccctcaattctctgaatgtctccagaaacaacctgaaggtgtttccagatccctgggcctgccctttgaaatgttgtaaagcttccagaaatgccctggaatgtctgccagacaaaatggctgtcttttggaaaaatcacctgaaggatgtggatttctcagaaaacgcactcaaagaagttcccctgggacttttccagcttgatgccctcatgttcttgaggttacaggggaaccagctggcggcacttccacctcaagagaagtggacctgcaggcagctcaaaaccctggatctctccagaaaccaacttggcaaaaatgaagatggactgaaaacgaagcgtattgcctttttcaccaccagaggtcgccagcgctccgggactgaggcagagacaactatggagttcagtgcatctctggtaaccattgtgttcctgtctaacaactgtaacctctgtgcatacacatgtgcagcaagtgtgctggaatttccggccttcctaagtgagtctttggaagtcctttgcctgaacgacaaccacctcgacacagtccctccctcggtttgcctactgaagagcttatcagagctctacttgggaaacaaccctggcctccgggagctccctcctgagctggggcagctgggcaacctctggcagctggacactgaagacctgaccatcagcaatgtgcctgcagaaatccaaaaagaaggccccaaagcaatgctgtcttacctgcgtgctcagctgcggaaagcggaaaagtgcaagctgatgaagatgatcatcgtgggtcccccgcgccagggcaagtccaccctcctggagatcttacagacggggagggccccccaggtggtgcatggagaggccaccatcaggaccaccaagtgggagctccagaggccggctggctcgagagccaaggtcaaggatggtctgcgtgcagagtccctgtgggttgagtccgtggagttcaacgtctgggacatcgggggaccggccagcatggccactgtcaaccagtgcttcttcacggacaaggccctgtacgtggtggtctggaacctggcgctgggggaggaggccgtggccaacctccagttctggctgctcaacatcgaggccaaggccccaaacgccgtggtgctggtggtcgggacgcacctggatttaattgaagccaagttccgtgtggaaaggattgcaacgctgcgtgcctatgtgctggcactctgccgctccccctccggctccagggccacaggcttcccagacatcaccttcaaacacttacatgagatttcctgcaagagcctggaaggtcaggaagggctgcgacagctgattttccacgtcacgtgcagcatgaaggacgtcggcagcaccatcggctgccagcgactggcagggcggctgatccccaggagctacctgagcctgcaggaggccgtgctggcagagcagcagcgccgcagccgggacgacgacgtgcagtacctgacggacaggcagctggagcagctggtggagcagacgcccgacaacgacatcaaggactacgaggacctgcagtcagccatcagcttcctcatagaaaccggcaccctgctccatttcccggacaccagccacggcctgaggaacctctacttcctcgaccctatttggctctccgaatgtctgcagaggatctttaatattaagggctctcggtcagtggccaagaatggggtgatcagagcagaagacctcaggatgctgctggtggggactggcttcacgcagcagacggaagagcagtacttccagttcctggccaagtttgagatcgccctgcccgtcgccaatgacagctacctcctgccccatctccttccatctaaacctggcctggacacccacggtatgcggcaccccacagccaacaccattcagagggtatttaagatgagcttcgttcccgttggcttctggcaaaggtttatagcacggatgctgatcagcctggcggagatggacctgcagctttttgaaaacaagaagaatactaaaagcaggaacaggaaagtcaccatttacagttttacaggaaaccagagaaatcgctgtagcacattcagagtgaaaagaaatcagaccatctattggcaggaagggctcctggtcacttttgatgggggctacctcagtgtggaatcttccgacgtgaactggaaaaagaagaaaagcggaggaatgaaaattgtttgccaatcagaagtgagggacttctcagccatggctttcatcacggaccacgtcaattccttgattgatcagtggtttcccgccctgacagccacagagagcgacgggacgccactcatggagcagtacgtgccctgcccggtctgcgagacagcctgggcccagcacacggaccccagtgagaaatcagaggatgtgcagtacttcgacatggaagactgtgtcctgacggccatcgagcgggacttcatctcctgccccagacacccggacctccccgtgccgctgcaggagctggtccctgaactgttcatgaccgacttcccggccaggctcttcctggagaacagcaagctggagcacagcgaggacgagggcagcgtcctgggccagggcggcagtggcaccgtcatctaccgggcccggtaccagggccagcctgtggccgtcaagcgcttccacatcaaaaaattcaagaactttgctaacgtaccggcagacaccatgctgaggcacctgcgggccaccgatgccatgaagaacttctccgagttccggcaggaggccagcatgctgcacgcgctgcagcacccctgcatcgtggcgctcatcggcatcagcatccacccgctctgcttcgccctggagctcgcgccgctcagcagcctcaacaccgtgctgtccgagaacgccagagattcttcctttatacccctgggacacatgctcacccaaaaaatagcctaccagatcgcctcgggcctggcctacctgcacaagaaaaacatcatcttctgtgacctgaagtcggacaacattctggtgtggtcccttgacgtcaaggagcacatcaacatcaagctatctgactacgggatttcgaggcagtcattccatgagggcgccctaggcgtggagggcactcctggctaccaggccccagagatcaggcctcgcattgtatatgatgagaaggtagatatgttctcctatggaatggtgctctacgagttgctgtcaggacagcgccctgcactgggccaccaccagctccagattgccaagaagctgtccaagggcatccgcccggttctggggcagccggaggaagtgcagttccggcgactgcaggcgctcatgatggagtgctgggacactaagccagagaagcgaccgctggccctgtcggtggtgagccagatgaaggacccgacttttgccaccttcatgtatgaactgtgctgtgggaagcagacagccttcttctcatcccagggccaggagtacaccgtggtgttttgggatggaaaagaggagtccaggaactacacggtggtgaacacagagaagggcctcatggaggtgcagaggatgtgctgccctgggatgaaggtgagctgccagctccaggtccagagatccctgtggacagccaccgagaattcctacctggtcttagcgggcctcgccgatgggcttgtggctgtgtttcccgtggtgcggggcaccccaaaggacagctgctcctacctgtgctcacacacagccaacaggtccaagttcagcatcgcggatgaagacgcacggcagaacccctacccagtgaaggccatggaggtggtcaacagcggctctgaggtctggtacagcaatgggccgggcctccttgtcatcgactgtgcctccctggagatctgcaggcggctggagccctacatggccccctccatggttacgtcagtcgtgtgcagctctgagggcagaggggaggaggtcgtctggtgcctggatgacaaggccaactccttggtgatgtaccactccaccacctaccagctgtgtgcccggtacttctgcggggtccccagccccctcagggacatgtttcccgtgcggcccttggacacggaacccccggcagccagccacacggccaacccaaaggtgcctgagggggactccatcgcggacgtgagcatcatgtacagtgaggagctgggcacgcagatcctgatccaccaggaatcactcactgactactgctccatgtcctcctactcctcatccccaccccgccaggctgccaggtccccctcaagcctccccagctccccagcaagttcttccagtgtgcctttctccaccgactgcgaggactcagacatgctacatacgcccggtgctgcctccgacaggtctgagcatgacctgacccccatggacggggagaccttcagccagcacctgcaggccgtgaagatcctcgccgtcagagacctcatttgggtccccaggcgcggtggagatgttatcgtcattggcctggagaaggattctggcgcccagcggggccgagtcattgccgtcttaaaagcccgagagctgactccgcatggggtgctggtggatgctgccgtggtggcaaaggacactgttgtgtgcacctttgaaaatgaaaacacagagtggtgcctggccgtctggaggggctggggcgccagggagttcgacattttctaccagtcctacgaggagctgggccggctggaggcttgcactcgcaagagaaggtaattcctgtggaatgactgtcacacatcagagctggctggcccggggctgcagcctgactcctctgccatcggcctctagttctccaaggacctagaagacagatggagttctcccctgaactccttgctgctaagaagtgctgagaagttactcgcctggcggtggctccagggttctctggttctctggagcagagttctctgaataccccatcccccaactgctgattttacagccccagggaagacagtggtatcaggctgggagcggcctcctctggcctcccccatcagtttgcaggagcaggggtgcaggatcctgttctgagctgggtcaaacaaagcagggccgggccttcctgccatccccaggtctcagatggaattacactagaggccctccgctgggaagcacttgaggtagggcaggaggggggctgtgacccctgccctttccccgccagagacctcaggctctcagcacattccacaggctcctgagtccccgaggcctgggccagcttgggcaagccaagatcagatgtctctgtgttcgggaaggtctccgtgtgggaaagcccttgggggatcccgggtgaggagtgttgccccatccagagaatgaatgagttcctttaagtgccaccgccagcaagcccagaggcacacattctgagtgcacccgcttagcctttacattcctctccaccgacaaaaggaaggggaaactcaatcagcaggacttcagaaagggccttgtgtttatagctttgtcaagtaaatttggacgcagctggaaacacaggcctgtttgttgcacata

SEQ ID NO: 8 - protein, 2014 aa, Homo sapiens
MAGMSQRPPSMYWCVGPEESAVCPERAMETLNGAGDTGGKPSTRGGDPAARSRRTEGIRAAYRRGDRGGARDLLEEACDQCASQLEKGQLLSIPAAYGDLEMVRYLLSKRLVELPTEPTDDNPAVVAAYFGHTAVVQELLESLPGPCSPQRLLNWMLALACQRGHLGVVKLLVLTHGADPESYAVRKNEFPVIVRLPLYAAIKSGNEDIAIFLLRHGAYFCSYILLDSPDPSKHLLRKYFIEASPLPSSYPGKTALRVKWSHLRLPWVDLDWLIDISCQITELDLSANCLATLPSVIPWGLINLRKLNLSDNHLGELPGVQSSDEIICSRLLEIDISSNKLSHLPPGFLHLSKLQKLTASKNCLEKLFEEENATNWIGLRKLQELDISDNKLTELPALFLHSFKSLNSLNVSRNNLKVFPDPWACPLKCCKASRNALECLPDKMAVFWKNHLKDVDFSENALKEVPLGLFQLDALMFLRLQGNQLAALPPQEKWTCRQLKTLDLSRNQLGKNEDGLKTKRIAFFTTRGRQRSGTEAETTMEFSASLVTIVFLSNNCNLCAYTCAASVLEFPAFLSESLEVLCLNDNHLDTVPPSVCLLKSLSELYLGNNPGLRELPPELGQLGNLWQLDTEDLTISNVPAEIQKEGPKAMLSYLRAQLRKAEKCKLMKMIIVGPPRQGKSTLLEILQTGRAPQVVHGEATIRTTKWELQRPAGSRAKVKDGLRAESLWVESVEFNVWDIGGPASMATVNQCFFTDKALYVVVWNLALGEEAVANLQFWLLNIEAKAPNAVVLVVGTHLDLIEAKFRVERIATLRAYVLALCRSPSGSRATGFPDITFKHLHEISCKSLEGQEGLRQLIFHVTCSMKDVGSTIGCQRLAGRLIPRSYLSLQEAVLAEQQRRSRDDDVQYLTDRQLEQLVEQTPDNDIKDYEDLQSAISFLIETGTLLHFPDTSHGLRNLYFLDPIWLSECLQRIFNIKGSRSVAKNGVIRAEDLRMLLVGTGFTQQTEEQYFQFLAKFEIALPVANDSYLLPHLLPSKPGLDTHGMRHPTANTIQRVFKMSFVPVGFWQRFIARMLISLAEMDLQLFENKKNTKSRNRKVTIYSFTGNQRNRCSTFRVKRNQTIYWQEGLLVTFDGGYLSVESSDVNWKKKKSGGMKIVCQSEVRDFSAMAFITDHVNSLIDQWFPALTATESDGTPLMEQYVPCPVCETAWAQHTDPSEKSEDVQYFDMEDCVLTAIERDFISCPRHPDLPVPLQELVPELFMTDFPARLFLENSKLEHSEDEGSVLGQGGSGTVIYRARYQGQPVAVKRFHIKKFKNFANVPADTMLRHLRATDAMKNFSEFRQEASMLHALQHPCIVALIGISIHPLCFALELAPLSSLNTVLSENARDSSFIPLGHMLTQKIAYQIASGLAYLHKKNIIFCDLKSDNILVWSLDVKEHINIKLSDYGISRQSFHEGALGVEGTPGYQAPEIRPRIVYDEKVDMFSYGMVLYELLSGQRPALGHHQLQIAKKLSKGIRPVLGQPEEVQFRRLQALMMECWDTKPEKRPLALSVVSQMKDPTFATFMYELCCGKQTAFFSSQGQEYTVVFWDGKEESRNYTVVNTEKGLMEVQRMCCPGMKVSCQLQVQRSLWTATENSYLVLAGLADGLVAVFPVVRGTPKDSCSYLCSHTANRSKFSIADEDARQNPYPVKAMEVVNSGSEVWYSNGPGLLVIDCASLEICRRLEPYMAPSMVTSVVCSSEGRGEEVVWCLDDKANSLVMYHSTTYQLCARYFCGVPSPLRDMFPVRPLDTEPPAASHTANPKVPEGDSIADVSIMYSEELGTQILIHQESLTDYCSMSSYSSSPPRQAARSPSSLPSSPASSSSVPFSTDCEDSDMLHTPGAASDRSEHDLTPMDGETFSQHLQAVKILAVRDLIWVPRRGGDVIVIGLEKDSGAQRGRVIAVLKARELTPHGVLVDAAVVAKDTVVCTFENENTEWCLAVWRGWGAREFDIFYQSYEELGRLEACTRKR

SEQ ID NO: 9 - DNA, 22 bp, Artificial Sequence
  Description of Artificial Sequence Primer
gtatgaactgtgctgtgggaag

SEQ ID NO: 10 - DNA, 23 bp, Artificial Sequence
  Description of Artificial Sequence Primer
gatgtaccactccaccacctacc

SEQ ID NO: 11 - DNA, 20 bp, Artificial Sequence
  Description of Artificial Sequence Primer
atggagacgcttaacggtgc

SEQ ID NO: 12 - DNA, 26 bp, Artificial Sequence
  Description of Artificial Sequence Primer
gttccctgtcatcgtgcgcttgcccc

SEQ ID NO: 13 - DNA, 25 bp, Artificial Sequence
  Description of Artificial Sequence Primer
ccagcaacaagttgtcccacctccc

SEQ ID NO: 14 - DNA, 25 bp, Artificial Sequence
  Description of Artificial Sequence Primer
ggatctctccagaaaccaacttggc

SEQ ID NO: 15 - DNA, 26 bp, Artificial Sequence
  Description of Artificial Sequence Primer
cctggagatcttacagacggggaggg

SEQ ID NO: 16 - DNA, 25 bp, Artificial Sequence
  Description of Artificial Sequence Primer
cgtcggcagcaccatcggctgccag

SEQ ID NO: 17 - DNA, 24 bp, Artificial Sequence
  Description of Artificial Sequence Primer
cctcctgccccatctccttccatc

SEQ ID NO: 18 - DNA, 25 bp, Artificial Sequence
  Description of Artificial Sequence Primer
gaaatcagaggatgtgcagtacttc

SEQ ID NO: 19 - DNA, 24 bp, Artificial Sequence
  Description of Artificial Sequence Primer
caccgtgctgtccgagaacgccag

SEQ ID NO: 20 - DNA, 24 bp, Artificial Sequence
  Description of Artificial Sequence Primer
ccttcatgtatgaactgtgctgtg

SEQ ID NO: 21 - DNA, 23 bp, Artificial Sequence
  Description of Artificial Sequence Primer
ccatggttacgtcagtcgtgtgc

SEQ ID NO: 22 - DNA, 23 bp, Artificial Sequence
  Description of Artificial Sequence Primer
ccgacaggtctgagcatgacctg

SEQ ID NO: 23 - DNA, 25 bp, Artificial Sequence
  Description of Artificial Sequence Primer
ttaccttctcttgcgagtgcaagcc

SEQ ID NO: 24 - DNA, 21 bp, Artificial Sequence
  Description of Artificial Sequence Primer
gagaactctgctccagagaac

SEQ ID NO: 25 - DNA, 24 bp, Artificial Sequence
  Description of Artificial Sequence Primer
cggatgacaacccagccgtggtgg

SEQ ID NO: 26 - protein, 10 aa, Artificial Sequence
  Description of Artificial Sequence Synthetic peptide substrate
KKLNRTLSVA